(19) United States
(12) Patent Application Publication
(10) Pub. No.: US 2004/0078838 A1
(43) Pub. Date: Apr. 22, 2004

(54) PROCESSES FOR INHIBITING AND FOR INDUCING FLOWER FORMATION IN PLANTS

(76) Inventors: BERND MULLER-ROBER, POTSDAM-EICHE (DE); VOLKER LANDSCHUTZE, BERLIN (DE); URSULA LA COGNATA, BERLIN (DE)

Correspondence Address: JAMES F HALEY JR, FISH & NEAVE, 1251 AVENUE OF THE AMERICAS, NEW YORK, NY, 10020

(21) Appl. No.: 08/702,718

(22) PCT Filed: Mar. 7, 1995
(86) PCT No.: PCT/EP95/00859

(30) Foreign Application Priority Data
Mar. 9, 1994 (DE) P 44 08 629.6
Sep. 22, 1994 (DE) P 44 35 366.9
Oct. 19, 1994 (DE) P 44 38 821.7

(51) Int. Cl. A01H 1/00; C12N 15/82

(52) U.S. Cl. 800/278

(57) ABSTRACT

Processes for inhibiting flower formation and processes for inducing flower formation in plants, and processes for improving the storage capability of storage organs of useful plants and processes for reducing the sprouting of tubers in tuberous plants are described.

Also described are DNA sequences which modify the activity of the citrate synthase of the plant upon integration into a plant genome, plasmids which contain these DNA sequences and transgenic plants in which modifications in the activity of the citrate synthase are brought about by introducing the DNA sequences.

The described DNA sequences are sequences from Solanum tuberosum, Nicotiana tabacum and Beta vulgaris which code for the enzyme citrate synthase.

The invention also describes transgenic potato plants in which an inhibition of flower formation, a reduction in the storage losses of the tubers and a reduction in the sprouting of the tubers comes about because of an inhibition of the citrate synthase activity, and transgenic potato plants in which a premature induction of flower production comes about because of the over-expression of a citrate synthase.

The present invention relates to processes for inhibiting flower formation and processes for inducing flower formation in plants, and to processes for improving the storage capability of storage organs of useful plants, and to processes for reducing the sprouting of tubers in tuberous plants. The present invention also relates to DNA sequences which code for plant citrate synthases and to new plasmids containing these DNA sequences, which, upon integration into a plant genome, modify the activity of the citrate synthase in the plant, and to transgenic plants in which modifications in the activity of the citrate synthase are brought about by introducing these DNA sequences.

Because of the continuously increasing demand for food, which results from the constantly growing world population, one of the tasks of biotechnology research is to endeavour to increase the yield of useful plants. One possibility of achieving this consists e.g. of modifying the flowering behaviour of agriculturally useful plants. Increasing the number of flowers is for example desirable with plants whose flowers, fruits or seeds are used agriculturally. Premature flower formation leads to a shortening of the period between sowing and flowering and can thus permit the cultivation of plants in climatic regions with shorter vegetation periods, or the application of two sowings within one vegetation period. Inhibiting flower formation can be advantageous in plants which multiply in predominantly vegetative manner, and can lead to an increased deposition of stored substances in storage organs. One example of such an agriculturally useful plant is the potato.

Targeted modification of the flowering behaviour in plants has however as yet not been possible since the process of inducing flower formation in plants is not yet very well understood as a whole. Various substances such as e.g. carbohydrates, cytokinins, auxin, polyamines and calcium are discussed as inducers of flower formation. Overall, however, the impression is created that flowering induction is a complex process in which several factors interact which have not as yet been unequivocally identified (Bernier et al. (1993) Plant Cell 5:1147-1155). To date, chemical substances have as a rule been used to modify flowering behaviour. Thus, it is e.g. known that inhibiting flower formation in the case of sugar cane, which leads to a considerable increase in the sugar yield, can be achieved by the exogenous application of different synthetic growth regulators (monuron, diuron, diquat). The use of such synthetic substances is, however, generally associated with a high expenditure and environmental risks which are difficult to assess.

It therefore appears desirable to provide processes which permit a targeted modification of the flowering behaviour, in particular inhibition or induction of the flower formation, in the case of various useful plants, whilst avoiding the use of synthetic substances.

It is therefore the object of the present invention to provide processes which permit plants to be produced whose flowering behaviour is modified, in particular plants which are inhibited in their flower formation, or plants which display premature flower formation and an increased amount of flowers.

The present invention describes genetic engineering processes in which a change occurs in the flowering behaviour of plants because of the modification of the activity of an enzyme which is involved in respiratory processes in the cells.

It was surprisingly found that a strong inhibiton of the citrate synthase activity in cells of potato plants leads to a complete inhibition of flower formation in these plants, and that increasing the citrate synthase activity in cells of transformed potato plants also leads to a modified flowering behaviour of the plants, in particular to premature flower formation and to an increased number of flowers.

To produce plants with a reduced citrate synthase activity, DNA sequences which code for enzymes with the enzymatic activity of a citrate synthase were isolated from different plant species. These are DNA sequences from plants of the Solanaceae family, in particular from Solanum tuberosum and Nicotiana tabacum, and sequences from plants of the Chenopodiacae family, in particular from sugar beet (Beta vulgaris). A subject of the invention are therefore DNA sequences from plants of the Solanaceae family, in particular the species Solanum tuberosum and Nicotiana tabacum, and of the Chenopodiaceae family, in particular the species Beta vulgaris, which code for enzymes having the enzymatic activity of a citrate synthase, and which, after integration into a plant genome, permit the formation of transcripts by which an endogenous citrate synthase activity can be suppressed, or the formation of transcripts by which citrate synthase activity in the cells can be increased. The invention relates in particular to DNA sequences which code for a protein having one of the amino sequences given in Seq ID No. 1, Seq ID No. 2 or Seq ID No. 3, or for a protein having an essentially identical amino acid sequence, and to DNA sequences which have one of the nucleotide sequences shown in Seq ID No. 1, Seq ID No. 2 or Seq ID No. 3, or an essentially identical nucleotide sequence. The invention also relates to derivatives of the sequences shown in Seq ID Nos. 1-3 which can be derived from these by insertion, deletion, substitution of one or more nucleotides or by recombination, and which code for proteins having the enzymatic activity of citrate synthase. Recombinant DNA molecules, e.g. plasmids, and bacteria containing these DNA sequences or sections or derivatives thereof are also a subject of the invention. The term “essentially identical” in relation to DNA and amino acid sequences means that the sequences in question have a high degree of homology and that there is functional and/or structural equivalence between the DNA sequences or amino acid sequences concerned. A high degree of homology is understood to be a sequence identity of at least 40%, preferably above 60% and particularly preferably above 80%. Sequences which are homologous to the sequences according to the invention and differ from the DNA sequence or amino acid sequence according to the invention at one or more positions are as a rule variations or derivatives of this sequence which represent modifications which perform the same function. They can however also be naturally occurring variations, for example sequences from other organisms, or mutations, where these mutations may have been caused naturally or were introduced through targeted mutagenesis. The variations can also be synthetically produced sequences. The proteins coded by the different variants of the DNA sequence according to the invention have certain common characteristics. These may include e.g. enzyme activity, immunological reactivity, conformation etc., and physical properties such as e.g. the mobility gel electrophoreses, chromatographic behaviour, sedimentation coefficients, solubility, spectroscopic properties, stability etc.

It was found that an inhibition of flower formation occurs in transformed plants when DNA sequences which code for a citrate synthase are introduced into plant cells and expressed in anti-sense orientation, which causes the citrate synthase activity in the cells to be reduced.

Within the scope of the present invention, inhibiting flower formation means that the transformed plants either no longer develop any flowers at all, develop fewer flowers than non-transformed plants or that some flowers do form but they do not develop into functional flowers. Inhibiting flower formation also means that the plants do indeed develop flowers, but that the latter are sterile and do not lead to the formation of seeds or fruits, or are capable of functioning to only a limited extent and lead to the formation of fewer seeds compared with wild-type plants. In particular, inhibiting flower formation means that male sterile flowers are formed or flowers in which the male reproductive organs form fertile pollen only to a small degree. The term means also that from the plants are formed flowers in which the female reproductive organs are absent, are not functional or are reduced in size compared with wild-type plants. Inhibiting flower formation also means that transformed plants, if they flower, flower later than non-transformed plants, as a rule several days later, preferably one to several weeks later, in particular 2 to 4 weeks later.

A subject of the invention is therefore the use of DNA sequences which code for a citrate synthase for inhibiting flower formation in plants, and the use of such sequences for the expression of a non-translatable mRNA which prevents the synthesis of endogenous citrate synthases in the cells.

The present invention also relates to a process for inhibiting flower formation in plants, characterized in that the citrate synthase activity in the cells of the plants is reduced, whereby this reduction is achieved preferably by inhibiting the expression of DNA sequences which code for citrate synthases.

Particularly preferred are processes in which flower formation inhibition is achieved by inhibiting the expression of endogenous citrate synthase genes through the use of anti-sense RNA.

The present invention relates in particular to processes for inhibiting flower formation in plants, characterized in that

a) a DNA which is complementary to a citrate synthase gene present in the cell is stably integrated into the genome of a plant cell,

b) this DNA is expressed constitutively or is inducible due to the combination with suitable elements controlling the transcription,

c) the expression of endogenous citrate synthase genes is inhibited because of an anti-sense effect and

d) plants are regenerated from the transgenic cells.

The expression of a DNA which is complementary to a citrate synthase gene present in the cell is as a rule achieved by integrating into the genome of the plants a recombinant double-stranded DNA molecule comprising an expression cassette having the following constituents and expressing it:

A) a promoter functional in plants,

B) a DNA sequence coding for citrate synthase which is fused to the promoter in anti-sense orientation, so that the non-coding strand is transcribed, and if necessary

C) a signal functional in plants for the transcription termination and polyadenylation of an RNA molecule.

Such DNA molecules are also a subject of the invention. The present invention provides such DNA molecules which contain the described expression cassettes in the form of the plasmid pKS-CSa (DSM 8880) which comprises the coding region for citrate synthase from potatoes, and of the plasmid TCSAS (DSM 9359) which comprises the coding region of citrate synthase from tobacco, the composition of which is described in Examples 3 and 8 respectively.

In principle, any promoter active in plants can be used as the promoter. The promoter is to ensure that the chosen gene is expressed in the plant. It is possible to use both those promoters which guarantee a constitutive expression in all tissues of the plant, such as e.g. the 35S promoter of the cauliflower mosaic virus, and those promoters which guarantee expression only in a certain tissue, at a certain time in plant development or at a time determined by external influences. The promoter can be homologous or heterologous in relation to the transformed plant. The use of tissue-specific promoters represents a preferred subject of the invention.

The DNA sequence which codes for a protein having the enzymatic activity of a citrate synthase can, in principle, originate from any chosen organism, preferably from plants. The sequence used originates preferably from the plant species which is used for the transformation, or from a closely related plant species. A preferred embodiment of the process discussed above provides that a DNA sequence which originates from a plant of the Solanaceae family or the Chenopodiaceae family, in particular from Solanum tuberosum, Nicotiana tabacum or Beta vulgaris is used for the DNA sequence which codes for a citrate synthase. Particularly preferred embodiments provide for the use of a DNA sequence which codes for a protein having one of the amino acid sequences given in SeqID No.1, SeqID No.2 or SeqID No.3 or an essentially identical amino acid sequence, in particular a DNA sequence which is identical or essentially identical to one of the DNA sequences given in SeqID No. 1, SeqID No. 2 or SeqID No. 3. Also, using standard processes and the already known DNA sequences which code for citrate synthases, other DNA sequences can be isolated from any organisms, preferably plants which code for proteins having the enzymatic activity of a citrate synthase. These sequences can also be used in the processes according to the invention.

The anti-sense orientation of the coding DNA sequence given in B) in relation to the promoter causes a non-translatable mRNA to form in the transformed plant cells which prevents the synthesis of an endogenous citrate synthase. Instead of the complete DNA sequences according to the invention given in SeqID No. 1, SeqID No.2 and SeqID No. 3, partial sequences thereof can also be used for the anti-sense inhibition. Sequences up to a minimum length of 15 bp can be used. However, an inhibiting effect is not excluded when shorter sequences are used either. Longer sequences between 100 and 500 base pairs are preferably used, for an efficient anti-sense inhibition, sequences having a length above 500 base pairs are used in particular. As a rule, sequences are used which are shorter than 5000 base pairs, preferably sequences which are shorter than 2500 base pairs. It is also possible to use DNA sequences which have a high degree of homology to the DNA sequences according to the invention, but which are not completely identical, in the process according to the invention. The minimum homology should be greater than approx. 65%. The use of sequences having homologies between 95 and 100% is to be preferred. DNA sequences can also be used which result from the sequences shown in SeqID No. 1, SeqID No. 2 or SeqID No. 3 by insertion, deletion or substitution without the inhibiting effect of the anti-sense sequence thereby being destroyed. The DNA fragments used for the construction of anti-sense constructs can also be synthetic DNA fragments which were produced using current DNA synthesis techniques.

The plants obtainable from the described process are also a subject of the invention, which are characterized in that they display a reduced citrate synthase activity in the cells as a result of the expression of an anti-sense RNA which is complementary to DNA sequences which code for a protein having the enzymatic activity of a citrate synthase. Such plants are also characterized in that they contain an expression cassette stably integrated into the genome, which comprises the following sequences:

A) a promoter functional in plants,

B) a DNA sequence coding for citrate synthase which is fused to the promoter in anti-sense orientation, so that the non-coding strand is transcribed, and if necessary

C) a signal functional in plants for the transcription termination and polyadenylation of an RNA molecule.

The plants are preferably the plants given above.

As is described in the embodiments taking the potato as an example, there occurs in potato plants, because of the reduction in the citrate synthase activity by means of an anti-sense effect, an inhibition of flower formation in transformed plants. In particular, transformed potato plants display more or less drastic phenotypes depending on the degree of reduction in the citrate synthase activity. A marked reduction in the citrate synthase leads to the complete inhibition of flower formation. Plants with a less marked inhibition do produce buds but these are not developed to functional flowers. Plants can also be produced which develop flowers, but whose female reproductive organs are not functional. Similar effects are observed with transgenic tobacco plants which show a reduction in the citrate synthase activity. Flowers are developed here also whose female reproductive organs are greatly reduced in size. The inhibition of flower formation via the reduction in the citrate synthase activity is not however only of interest for potatoes or tobacco, but should be of wider significance for plant breeding and agriculture. E.g. the possibility can be cited of achieving a chronologically determined flower induction or inhibition by combining the DNA sequences according to the invention with exogenously regulatable control elements. This can play a role in the prevention of frost damage. The processes according to the invention can be used both on dicotyledons as well as on monocotyledons. Plants which are of particular interest are useful plants such as types of grain (e.g. rye, wheat, corn, oats, barley, maize, rice etc.), types of fruit (e.g. apricots, peaches, apples, plums etc.), types of vegetable (e.g. tomatoes, broccoli, asparagus etc.), ornamental plants or other economically interesting types of plants (e.g. potatoes, tobacco, rapeseed, soya beans, sunflowers, sugar cane etc.). The use of the present invention in particular with sugar beet is of particular interest, since here “shooting” can be prevented by inhibiting flower formation. Since shooting is induced by low temperatures, the seeds are planted relatively late (in April/May) in order to prevent shooting. By inhibiting the citrate synthase in sugar cane, a reduction in shooting would be achieved. This permits the sugar beet seeds to be sown earlier which then leads to an increased yield because of the extended vegetation period.

In addition to inhibiting flower formation, in transformed potato plants which display a reduced citrate synthase activity in the cells, a reduced sprouting of the tubers and a reduced respiration in cells of the tubers was observed, compared with non-transformed plants. This leads to lower storage losses and an improved storage capability of the tubers. The process according to the invention is therefore also suitable for producing plants with an improved storage capability of the storage organs, whereby improved storage capability is understood within the context of this invention to mean that the stored storage organs of transformed plants show smaller losses of fresh and dry weight after a period of storage, compared with those of non-transformed plants. Storage organs are understood to be typical harvestable organs of plants, such as seeds, fruits, tubers and beets.

The process is suitable in particular for producing transgenic potato plants whose tubers have an improved storage capability, smaller storage losses and reduced sprouting of tubers compared with wild-type plants. Reduced sprouting of tubers means that the tubers of transformed plants form sprouts which have a lower fresh and dry weight compared with sprouts of non-transformed plants. The commercial benefits of these effects are obvious.

A subject of the invention are therefore also processes for improving the storage capability of storage organs in plants, characterized in that the citrate synthase activity in the cells of the plants is reduced, this reduction preferably being achieved by inhibiting the expression of DNA sequences which code for citrate syntheses.

Particularly preferred are processes in which the citrate synthase activity is reduced by inhibiting the expression of endogenous citrate synthase genes through the use of anti-sense RNA.

The present invention relates in particular to processes for improving the storage capability of storage organs in plants, characterized in that

a) a DNA which is complementary to a citrate synthase gene present in the cell is stably integrated into the genome of a plant cell,

b) this DNA is expressed constitutively or inductively by combination with suitable elements controlling the transcription,

c) the expression of endogenous citrate synthase genes is inhibited by an anti-sense effect and

d) plants are regenerated from the transgenic cells.

Such processes can be used on all types of plants which develop storage organs, preferably on agricultural useful plants and particularly preferably on types of grain (rye, barley, wheat, maize, rice etc.), types of fruit, types of vegetable, on plants which develop tubers such as e.g. potatoes or manioc, and on plants which develop beet as storage organs, in particular sugar beet.

A subject of the invention are also processes for the production of transgenic tuberous plants whose tubers display reduced sprouting, characterized in that the citrate synthase activity in the cells of the plants is reduced, this reduction preferably being achieved by inhibiting the expression of DNA sequences which code for citrate synthases.

Particularly preferred are processes in which the reduction in the citrate synthase activity is achieved by inhibiting the expression of endogenous citrate synthase genes through the use of anti-sense RNA.

The present invention relates in particular to processes for the production of transgenic tuberous plants whose tubers display reduced sprouting, characterized in that

a) a DNA which is complementary to a citrate synthase gene present in the cell is stably integrated into the genome of a plant cell,

b) this DNA is expressed constitutively or inductively by combination with suitable elements controlling the transcription,

c) the expression of endogenous citrate synthase genes is inhibited because of an anti-sense effect and

d) plants are regenerated from the transgenic cells.

Such processes can preferably be used for the production of transgenic potato and manioc plants.

What has already been stated above for the process for inhibiting flower production also applies to the various possibilities in the embodiments of the given processes, in particular for the choice and length of the DNA sequence used which codes for a citrate synthase, and to the choice of promoter.

As an alternative to reducing the citrate synthase activity in plant cells using an anti-sense effect, the reduction can also be achieved by introducing a DNA sequence which codes for a ribozyme which specifically cleaves transcripts of endogenous citrate synthase genes in endonucleolytic manner. Ribozymes are catalytically active RNA molecules which are able to cleave RNA molecules at specific target sequences. Using genetic engineering methods it is possible to modify the specificity of ribozymes. There are different classes of ribozymes. For practical application with the aim of cleaving the transcript of a certain gene in targeted manner, representatives of two different groups of ribozymes are preferably used. The first group comprises ribozymes which are to be assigned to the GroupI-intron-ribozymes. The second group comprises ribozymes which have as a characteristic structural feature a so-called “hammerhead” motif. The specific recognition of the target RNA molecule can be modified by changing the sequences which flank this motif. Via base pairing with sequences in the target molecule, these sequences determine the site at which the catalytic reaction and therefore cleavage of the target molecule takes place. Since the sequence requirements for an efficient cleavage are extremely low, it therefore appears possible in principle to develop specific ribozymes for practically any RNA molecule.

Genetically modified plants whose citrate synthase activity is drastically reduced can therefore also be produced by introducing and expressing a recombinant double-stranded DNA molecule in plants which comprises:

a) a promoter functional in plants

b) a DNA sequence which codes for a catalytic domain of a ribozyme and which is flanked by DNA sequences which are homologous to sequences of the target molecule, and, if necessary,

c) a signal, functional in plants, for the transcription termination and polyadenylation of an RNA molecule.

Coming into consideration for the sequence under b) are e.g. the catalytic domain of the satellite DNA of the SCMo virus (Davies et al., 1990, Virology, 177:216-224) or that of the satellite DNA of the TobR virus (Steinecke et al., 1992, EMBO J., 11:1525-1530; Haseloff and Gerlach, 1988, Nature 334:585-591). The DNA sequences which flank the catalytic domain are formed of DNA sequences which are homologous to the sequences of endogenous citrate synthase genes. The same as was already stated above for the construction of anti-sense structures applies to the sequences given in a) and c).

A further aspect of the present invention consists in the expression of DNA sequences which code for proteins having the enzymatic activity of a citrate synthase in sense orientation in plant cells in order to increase the citrate synthase activity. For this, a DNA sequence coding for citrate synthase is fused in sense orientation to a promoter, i.e. the 3′-end of the promoter is linked to the 5′-end of the coding DNA sequence. This leads to the expression of an mRNA coding for citrate synthase and consequently to an increased synthesis of this enzyme.

It was now surprisingly found that, as a result of the increase in the citrate synthase activity in cells of transformed plants, a modification of flowering behaviour occurs compared with non-transformed plants. In particular, flower formation is induced. Within the scope of the present invention, the following are understood by this:

a) a premature flower formation (this means in this connection that transformed plants flower earlier compared with non-transformed plants, as a rule a few days earlier, preferably one to several weeks earlier) and/or

b) an enhanced flower formation (this means in this connection that transformed plants produce more flowers, preferably at least 10% more flowers, compared with non-transformed plants).

Such an effect is desirable in a series of cultivated and useful plants such as types of vegetables, e.g. tomatoes, paprika, pumpkin, melons, gherkins, courgettes, rapeseed, types of grain, maize or cotton and in various ornamental plants.

A further subject of the present invention is therefore the use of DNA sequences which code for proteins having the enzymatic activity of a citrate synthase, for inducing flower formation in plants, and processes for inducing flower formation in plants, characterized in that the citrate synthase activity in the cells of the plants is increased. The citrate synthase activity is increased preferably by introducing a recombinant DNA molecule into plant cells which comprises the coding region for a citrate synthase and which leads to the expression of a citrate synthase in the transformed cells.

Such processes preferably comprise the following steps:

a) stably integrating a DNA, which is of homologous or heterologous origin and which codes for a protein having citrate synthase activity, into the genome of a plant cell,

b) expressing this DNA constitutively or inductively by combining with suitable elements controlling the transcription,

c) thereby increasing the citrate synthase activity in the cells and

d) regenerating plants from the transgenic cells.

The expression of a DNA which codes for a protein having the enzymatic activity of a citrate synthase is as a rule achieved by integrating a recombinant double-stranded DNA molecule comprising an expression cassette having the following constituents into the genome of the plants and expressing it:

A) a promoter functional in plants,

B) a DNA sequence coding for citrate synthase which is fused to the promoter in sense orientation, and if necessary

C) a signal functional in plants for the transcription termination and polyadenylation of an RNA molecule.

Such DNA molecules are also a subject of the invention. The present invention provides those DNA molecules which contain such expression cassettes, in the form of the plasmid pHS-mCS, which comprises the coding region for citrate synthase from S. cerevisiae, and of the plasmid pEC-mCS, which comprises the coding region of citrate synthase from E. coli.

The DNA sequences given in point a) of the process, which code for citrate synthase, can be of both homologous or native and heterologous or foreign origin in relation to the host plant to be transformed. They can be of pro- as well as eukaryotic origin. DNA sequences coding for citrate synthase from the following organisms are for example known: Bacillus subtilis (U05256 and U05257), E. coli (V01501), R. prowazekii (M17149), P. aeruginosa (M29728), A. anitratuzm (M33037) (see Schendel et al. (1992) Appl. Environ. Microbiol. 58:335-345 and references contained therein), Haloferax volcanii (James et al. (1992) Biochem. Soc. Trans. 20:12), Arabidopsis thaliana (Z17455) (Unger et al. (1989) Plant Mol. Biol. 13:411-418), B. coagulans (M74818), C. burnetii (M36338) (Heinzen et al. (1991) Gene 109:63-69), M. smegmatis (X60513), T. acidophilum (X55282), T. thermophila (D90117), pig (M21197) (Bloxham et al. (1981) Proc. Natl. Acad. Sci. 78:5381-5385), N. crassa (M84187) (Ferea et al. (1994), Mol. Gen. Genet. 242:105-110) and S. cerevisiae (Z11113, Z23259, M14686, M54982, X00782) (Suissa et al. (1984) EMBO J. 3:1773-1781). The numbers in brackets give in each case the accession numbers under which these sequences are accessible in the GenEMBL data bank. The sequences can be isolated from the said organisms by means of current molecular biology techniques or they can be produced synthetically.

A preferred embodiment of the process according to the invention provides for the use of DNA sequences which code for citrate synthases which, compared with citrate synthases normally occurring in plants, are deregulated or unregulated, i.e. are not regulated in their enzymatic activity by regulation mechanisms which influence the activity of the citrate synthase in plant cells. Deregulated means in particular that these enzymes are not inhibited to the same degree by the inhibitors or activated by the activators which normally inhibit or activate plant citrate synthases.

Unregulated citrate synthases are understood within the scope of this invention to be citrate synthases which are not subject to regulation by inhibitors or activators in plant cells.

Prokaryotic, in particular bacterial, DNA sequences are preferably used which code for citrate synthases since they have the advantage that the proteins which are coded by these sequences are subject to no regulation or only weak regulation in plant cells. It is thereby possible that an increase in citrate synthase activity occurs through expression of an additional citrate synthase in plant cells.

In a preferred embodiment of the described process, DNA sequences from E. coli are used which code for a protein with citrate synthase activity, in particular the gene glt A (Sarbjit et al., 1983, Biochemistry 22:5243-5249).

A further preferred embodiment of the process according to the invention provides for the use of DNA sequences from Saccharomyces cerevisia which code for citrate synthase, in particular the use of the DNA sequences described by Suissa et al. (1984, EMBO J. 3:1773-1781).

In cases where plant DNA sequences are used, DNA sequences are preferably used which code for a protein having one of the amino acid sequences given in Seq ID No. 1 or Seq ID No. 2 or Seq ID No. 3 or an essentially identical amino acid sequence. Shorter DNA sequences can also be used which code only for parts of the amino acid sequences given in Seq ID No. 1 Seq ID No. 2 or Seq ID No. 3, provided that the resulting protein is guaranteed to have the enzymatic activity of a citrate synthase.

A particularly preferred embodiment consists of a process in which the DNA sequence coding for a citrate synthase activity comprises the nucleotide sequence given in Seq ID No. 1 or Seq ID No. 2 or Seq ID No. 3, or an essentially identical nucleotide sequence or a part thereof, this part being long enough to code for a protein which displays citrate synthase activity.

In addition, with the help of standard processes using DNA the already known sequences which code for citrate synthases, other DNA sequences can be isolated from any organisms, preferably from plants and prokaryotic organisms, which code for proteins having the enzymatic activity of a citrate synthase. These sequences can also be used in the processes according to the invention.

Using the process according to the invention, the citrate synthase activity can in principle be increased in every compartment of a transformed cell. There will preferably be an increase in the activity in the mitochondria, the glyoxysomes or the cytosol. In order to guarantee localisation of the citrate synthase in a certain compartment of the transformed cells, the coding sequence must be linked to the sequences necessary for localisation into the corresponding compartment. Such sequences are known. For localising the citrate synthase in the mitochondria it is for example necessary that the expressed protein has at the N-terminus a mitochondrial targeting sequence (signal sequence) which guarantees the transportation of the protein expressed in the cytosol into the mitochondria. If the gene used does not already comprise a sequence which codes for a signal peptide, such a sequence must be introduced using genetic engineering methods. A sequence which codes for a mitochondrial targeting sequence is for example known from Braun et al. (1992, EMBO J. 11: 3219-3227). The sequence must be linked to the coding region in such a way that the polypeptide coded by the target sequence lies in the same reading frame as the subsequent DNA sequence coding for citrate synthase. If bacterial DNA sequences are used which code for a citrate synthase, then all 5′-non-translated regions are preferably removed in these. If the bacterial enzyme has signal sequences, then these are preferably replaced by plant signal sequences.

The same as was already stated above in connection with the processes according to the invention for inhibiting flower formation applies to the choice of suitable transcriptional regulatory sequences, in particular promoters for expressing the DNA sequence which codes for citrate synthase and termination signals.

The described process can be used both on dicotyledons and on monocotyledons. Plants which are of particular interest are useful plants such as types of grain (e.g. rye, wheat, corn, barley, maize etc.), types of fruit (e.g. apricots, peaches, apples, plums etc.), types of vegetables (e.g. tomatoes, paprika, pumpkin, melons, gherkins, courgettes, broccoli, asparagus etc.), ornamental plants or other economically interesting types of plants (e.g. tobacco, rapeseed, soya beans, cotton, sunflowers etc.).

A subject of the invention are also the plants obtainable from the described process which are characterized in that they display an increased citrate synthase activity in the cells because of the additional expression of a DNA sequence which codes for a protein having the enzymatic activity of a citrate synthase. Such plants are also characterized in that they contain an expression cassette stably integrated into the genome, which comprises the following sequences:

A) a promoter functional in plants,

B) a DNA sequence coding for citrate synthase which is fused to the promoter in sense orientation, and if necessary

C) a signal functional in plants for the transcription termination and polyadenylation of an RNA molecule.

The plants are preferably those listed above.

By combining the DNA sequences according to the invention in the described processes for inhibiting or for inducing flower formation with exogenously regulatable control elements for the transcription, e.g. temperature-induced promoters, there also exists the possibility of chronologically determined flowering induction or flowering inhibition, depending on whether the DNA sequence is fused to the promoter in sense or anti-sense orientation. Thus, promoters are known inter alia for a specific expression in flower buds (Huisjer et al. (192) EMBO J. 11:1239-1249) or in photosynthetically active tissues, e.g. the ST-LS1 promoter (Stockhaus et al., 1989, EMBO J.8:2445-2451). To prevent the sprouting of potato tubers, and the storage losses through metabolization of the storage substances, appropriate promoters are those which ensure an activation of the transcription in the storage organs. In the case of potatoes, promoters are known which ensure an expression specifically in the tuber, e.g. promoters of class I patatin genes. An example is the promoter of the patatin gene B33 of Solanum tuberosum (Rocha-Sosa et al., 1989, EMBO J. 8:23-29). Through combination with exogenously regulatable control elements, for example wound-inducible or temperature-regulated promoters, the problem of vegetative multiplication in the case of potato plants whose tubers do not sprout upon inhibition of the citrate synthase can be solved. In the case of sugar beet, in analogous manner by using a beet-specific promoter, respiration can be reduced and consequently a yield loss through sugar degradation in the beet can be lessened.

For preparing the introduction of foreign genes into higher plants, a large number of cloning vectors are available which contain a replication signal for E. coli and a marker gene for the selection of transformed bacterial cells. Examples of such vectors are pBR322, pUC series, M13mp series, pACYC184 etc. The desired sequence can be introduced into the vector at a suitable restriction cleavage site. The plasmid obtained is used for the transformation of E. coli cells. Transformed E. coli cells are grown in a suitable medium, then harvested and lysed. The plasmid is recovered. Restriction analyses, gel electrophoreses and other biochemical-molecular biology methods are generally used as analysis method to characterize the plasmid DNA obtained. After each manipulation, the plasmid DNA can be cleaved and joined to other DNA sequences. Each plasmid DNA sequence can be cloned in the same or other plasmids. A multitude of techniques are available for the introduction of DNA into a plant host cell. These techniques include the transformation of plant cells with T-DNA using Agrobacterium tumefaciens or Agrobacterium rhizogenes as transformation agents, the fusion of protoplasts, injection, the electroporation of DNA, the introduction of DNA using the bio-ballistic method and other possibilities. For the injection and electroporation of DNA into plant cells, no special requirements as such are placed on the plasmids used. Simple plasmids such as e.g. pUC derivatives can be used. If, however, whole plants are to be regenerated from cells transformed in this manner, the presence of a selectable marker gene is necessary. According to the method of introducing desired genes into the plant cell, other DNA sequences can be necessary. If e.g. the Ti- or Ri-plasmid is used for the transformation of the plant cell, then at least the right border, although frequently the right and left border, of the Ti- and Ri-plasmid T-DNA must be joined as flanking region to the genes to be introduced.

If agrobacteria are used for the transformation, the DNA to be introduced must be cloned in special plasmids, either into an intermediate vector or into a binary vector. The intermediate vectors can be integrated into the Ti- or Ri-plasmid of the agrobacteria by homologous recombination because of sequences which are homologous to sequences in the T-DNA. This also contains the vir region necessary for the transfer of the T-DNA. Intermediate vectors cannot replicate in agrobacteria. By means of a helper plasmid, the intermediate vector can be transferred into Agrobacterium tumefaciens (conjugation). Binary vectors can replicate both in E. coli and in agrobacteria. They contain a selection marker gene and a linker or polylinker which are framed by the right and left T-DNA border regions. They can be transformed directly into the agrobacteria (Holsters et al. (1978) Mol. Gen. Genet. 163:181-187). The agrobacterium serving as host cell has to contain a plasmid which carries a vir region. The vir region is necessary for transferring the T-DNA into the plant cell. Additional T-DNA can be present. The agrobacterium transformed in this way is used for the transformation of plant cells. The use of T-DNA for the transformation of plant cells has been intensively investigated and adequately described in EP 120516; Hoekema, in: The Binary Plant Vector System Offsetdrukkerij Kanters B. V., Alblasserdam (1985), Chapter V; Fraley et al., Crit. Rev. Plant. Sci., 4: 1-46 and An et al. (1985) EMBO J. 4: 277-287.

To transfer the DNA into the plant cell, plant explants can be expediently co-cultivated with Agrobacterium tumefaciens or Agrobacterium rhizogenes. Whole plants can then be regenerated from the infected plant material (e.g. pieces of leaves, stem segments, roots or also protoplasts or suspension-cultivated plant cells) in a suitable medium which can contain antibiotics or biocides for the selection of transformed cells. The plants thus obtained can then be investigated for the presence of the introduced DNA.

Once the introduced DNA is integrated in the genome of the plant cell, it is as a rule stable there and is retained even in the successors of the cell originally transformed. It normally contains a selection marker which makes the transformed plant cell resistant to a biocide or an antibiotic such as kanamycin, G 418, bleomycin, hygromycin or phosphinothricin etc. The individually selected marker should therefore permit to distinguish transformed cells from cells which lack the introduced DNA. The transformed cells grow within the plant in the usual manner (see also McCormick et al. (1986) Plant Cell Reports 5:81-84). The resulting plants can be grown normally and be crossed with plants which have the same transformed genetic code or other genetic codes. The hybrid individuals resulting therefrom have the appropriate phenotypic properties. Two or more generations should be grown in order to ensure that the phenotypic feature is stably retained and inherited. Seeds should also be harvested in order to ensure that the corresponding phenotype or other characteristics are retained.

In addition to the uses already mentioned, the DNA sequences according to the invention can also be introduced into plasmids which permit a mutagenesis or a sequence modification through insertion, deletion or recombination of DNA sequences in prokaryotic or eukaryotic systems. The sequences can also be provided with control elements for expression in pro- and eukaryotic cells and be introduced into the appropriate cells.

The DNA sequences according to the invention can also be used to isolate from the genome of plants of different species homologous sequences which also code for a citrate synthase. In this context, homology means a sequence identity of at least 60%, preferably above 80% and in particular above 95%. The identification and isolation of such sequences is carried out according to standard processes (see e.g. Sambrook et al., 1989, Molecular Cloning, A Laboratory Manual, 2nd. ed. Cold Spring Harbor Laboratory Press, Cold Spring Harbour, N.Y.). With these sequences, constructions for the transformation of plants or microorganisms can in turn be produced.

Deposit

The plasmids produced and used within the scope of the present invention were deposited at the Deutsche Sammlung von Mikroorganismen (German Collection of Microorganisms) (DSM) in Brunswick, Federal Republic of Germany, which is recognised as an international depository, in accordance with the requirements of the Budapest Treaty on the International Recognition of the Deposit of Microorganisms for the Purposes of Patent Procedure. On 28.12.1993 the following plasmids were deposited at the German Collection of Microorganisms (DSM) (Deposit number):

Plasmid pPCS (DSM 8879)

Plasmid pKS-CSa (DSM 8880)

On 10.08.1994 the following plasmids were deposited at the German Collection of Microorganisms (Deposit number):

Plasmid pTCS (DSM 9357)

Plasmid pSBCS (DSM 9358)

Plasmid TCSAS (DSM 9359)

Abbreviations used

| | |
|---|---|
| BSA | bovine serum albumin |
| EDTA | (ethylene dinitrilo) tetraacetic acid |
| 50x Denhardt solution | 5 g Ficoll (type 400, Pharmacia) |
| | 5 g polyvinyl pyrrolidone |
| | 5 g bovine serum albumin (Fraction V, |
| | Sigma) |
| | to 500 ml with H₂O |
| FADH₂ | flavin-adenine-dinucleotide, reduced |
| MOPS | 3-(N-morpholino)-propanesulphonic acid |
| NADH | b-nicotiflamide adenine dinucleotide, |
| | reduced |
| PCR | polymerase chain reaction |
| PMSF | phenyl methyl sulphonyl fluoride |
| SCMo-virus | “subterranean clover mottle virus” |
| SDS | sodium dodecyl sulphate |
| 20x SSC | 175.3 g NaCl, 88.2 g sodium citrate |
| | to 1000 ml with H₂O, pH 7.0 with 10 N |
| | NaOH |
| TobR-virus | “tobacco ringspot virus” |
| Trizin | N-tris(hydroxymethyl) methyl glycin |

### DESCRIPTION OF THE FIGURES

FIG. 1 shows the plasmid pPCS (DSM 8879)

The feint line corresponds to the sequence of pBluescript KS. The bold line represents the cDNA which codes for citrate synthase from Solanum tuberosum. Restriction cleavage sites of the insertion are shown.

FIG. 2 shows the plasmid pKS-CSa (DSM 8880)

Structure of the plasmid:

A=Fragment A: CaMV 35S promoter, nt 6909-7437 (Franck et al. (1980) Cell 21:285-294)

B=Fragment B: cDNA from Solanum tuberosum coding for citrate synthase; BamHI/SalI-fragment from pPCS, approx. 1900 bp Orientation to the promoter: anti-sense

C=Fragment C: nt 11748-11939 of the T-DNA of the Ti plasmid pTiACH5 (Gielen et al. (1984) EMBO J. 3:835-846)

FIG. 3 shows the plasmid pSBCS (DSM 9358)

The feint line corresponds to the sequence of pBluescript SK. The bold line represents the cDNA which codes for citrate synthase from Beta vulgaris L. Restriction cleavage sites of the insertion are shown.

FIG. 4 shows the plasmid pTCS (DSM 9357)

The feint line corresponds to the sequence of pBluescript SK. The bold line represents the cDNA which codes for citrate synthase from Nicotiana tabacum. Restriction cleavage sites of the insertion are shown.

FIG. 5 shows the plasmid TCSAS (DSM 9359)

Structure of the plasmid:

A=Fragment A: CaMV 35S promoter, nt 6909-7437 (Franck et al. (1980) Cell 21:285-294)

B=Fragment B: cDNA from Nicotiana tabacum, coding for citrate synthase; BamHI/SalI fragment from pTCS, approx. 1800 bp Orientation to the promoter: anti-sense

C=Fragment C: nt 11748-11939 of the T-DNA of the Ti plasmid pTiACH5 (Gielen et al. (1984) EMBO J. 3:835-846)

FIG. 6 shows the result of a Northern Blot experiment.

2 μg poly(A⁺)-mRNA from different transgenic potato plants (lanes 4-8) and three non-transformed potato plants (lanes 1-3) were used in each case for the analysis.

lanes 1, 2, and 3: Wild-type Solanum tuberosum cv. Désirée

lane 4: transgenic potato line T6

lane 5: transgenic potato line T21

lane 6: transgenic potato line T29

lane 7: transgenic potato line T50

lane 8: transgenic potato line T55

For the hybridization, the radioactively labelled cDNA of the citrate synthase from potatoes was used.

FIG. 7 shows transgenic potato plants of the line T6 (Nos. 3 and 4) and T29 (Nos. 5 and 6) which were transformed with the plasmid pKS-CSa, compared with wild-type plants (Nos. 1 and 2). The plants were kept in a greenhouse at 60% humidity, at 22° C. for 16 h in the light and at 15° C. for 8 h in the dark.

FIG. 8 shows, as a diagram, the number of flowers produced in potato plants which had been transformed with the plasmid pKS-CSa, compared with wild-type plants. The number of plants with fully-developed open flowers during a flowering period is shown. 5 transgenic lines (T6, T21, T29, T50 and T55) are compared with wild-type plants. The transgenic line T21 is a transgenic line which displays no inhibition of the citrate synthase (100% citrate synthase activity). During the period of investigation, no plant of the line T29 developed flowers and the plants of the lines T6 and T50 begin to flower only approx. 3 weeks later than wild-type plants. Day 1 stands for the first day on which clearly visible buds were to be seen on the plants.

wt=wild-type

t6, t21, t29, t50, t55 =transgenic lines T6, T21, T29, T50 and T55.

FIG. 9 shows longitudinal sections through flower buds of wild type plants and transgenic plants of the line T29 for comparison

A: flower bud of a wild-type plant

B: Enlargement of the ovarian structure of the bud from A

C: Flower bud of a plant of the transgenic line T29

D: Enlargement of the ovarian structure of the bud from C

an: anthers

ov: ovary

pe: petals

se: sepals

The tissue damage in the ovaries of transgenic plants is clearly visible.

FIG. 10 shows the germinating behaviour of tubers of potato plants, of line T6 (left) which had been transformed with the plasmid pKS-CSa, compared with tubers of wild-type plants (right). The tubers had been stored for 9 months in the dark at room temperature.

FIG. 11 shows a flower of a tobacco plant which had been transformed with the plasmid TCSAS (left), compared with a flower of a non-transformed tobacco plant (right). The pistil of the flower of the transformed plant is much shorter than the pistil of the flower of the wild-type plant.

FIG. 12 shows the plasmid pHS-mCS

Structure of the plasmid:

A=Fragment A: CaMV 35S promoter, nt 6909-7437 (Franck et al. (1980) Cell 21:285-294)

B=Fragment B: 99 bp long DNA fragment which codes for the mitochondria targeting sequence of the matrix processing peptidase (MPP) (Braun et al., 1992, EMBO J. 11:3219-3227)

C=Fragment C: DNA sequence from Saccharomyces cerevisiae coding for citrate synthase (nucleotides 376-1818; Suissa et al., 1984, EMBO J. 3:1773-1781) orientation to the promoter: sense

D=Fragment D: nt 11748-11939 of the T-DNA of the Ti plasmid pTiACH5 (Gielen et al. (1984) EMBO J. 3:835-846)

FIG. 13 shows two transgenic potato plants of two independent lines which had been transformed with the plasmid pHS-mCS (middle and right), compared with a wild-type plant (left). The plants were kept in a greenhouse and are approx. 6 weeks old. Whilst the wild type plant has still formed no inflorescence, the two transgenic tobacco plants already have in each case a fully developed inflorescence.

FIG. 14 shows the plasmid pEC-mCS

Structure of the plasmid:

A=Fragment A: CaMV 35S promoter, nt 6909-7437 (Franck et al. (1980) Cell 21:285-294)

B=Fragment B: 99 bp long DNA fragment which codes for the mitochondria targeting sequence of the matrix processing peptidase (MPP) (Braun et al., 1992, EMBO J. 11:3219-3227)

C=Fragment C: DNA sequence from E. coli coding for citrate synthase (nucleotides 306-1589; Sarbjit et al., 1983, Biochemistry 22: 5244-5249) orientation to the promoter: sense

D=Fragment D: nt 11748-11939 of the T-DNA of the Ti plasmid pTiACH5 (Gielen et al. (1984) EMBO J. 3:835-846)

To provide a better understanding of the following examples, the most important processes used are explained below.

1. Cloning Procedure

For the cloning in E. coli the vector pBluescriptKS and the vector pBluescriptSK (Stratagene, USA) were used.

For the plant transformation the gene constructions were cloned into the binary vector pBinAR.

2. Bacterial Strains

For the pBluescript vectors and for the pBinAR vectors E. coli strain DH5α (Bethesda Research Laboratories, Gaithersburg, USA) was used. For the in vivo excision the E. coli strain XL1-Blue was used.

The transformation of the plasmids into the potato plants and tobacco plants was carried out using the Agrobacterium tumefaciens strain C58C1 (Rocha-Sosa et al. (1989) EMBO J. 8:23-29).

3. Transformation of Agrobacterium tumefaciens

The DNA was transferred by direct transformation according to the methods of Höfgen & Willmitzer (1988, Nucleic Acids Res. 16:9877). The plasmid DNA of transformed agrobacteria was isolated according to the Birnboim & Doly method (1979, Nucleic Acid Res. 7:1513-1523) and analyzed by means of gel electrophoresis after suitable restriction cleavage.

4. Transformation of Potatoes

Ten small scalpel-scored leaves of a potato sterile culture (Solanum tuberosum L. cv. Desiree) were placed in 10 ml MS medium (Murashige & Skoog (1962) Physiol. Plant. 15: 473) with 2% saccharose, which contained 50 μl of an Agrobacterium tumefaciens overnight culture, grown under selection. After 3-5 minutes' gentle shaking, they were further incubated for 2 days in the dark. After that, the leaves were placed on MS medium with 1.6% glucose, 5 mg/l naphthyl acetic acid, 0.2 mg/l benzyl aminopurine, 250 mg/l Claforan, 50 mg/l kanamycin, and 0.80% bactoagar for callus induction. After 1 week's incubation at 25° C. and 3000 Lux the leaves were placed on MS medium with 1.6% glucose, 1.4 mg/l zeatin ribose, 20 mg/l naphthyl acetic acid, 20 mg/l gibberellic acid, 250 mg/l Claforan, 50 mg/l kanamycin, and 0.80% bactoagar for shoot induction.

5. Transformation of Tobacco

An overnight culture of the corresponding Agrobacterium tumefaciens clone was centrifuged off (6500 rpm; 3 min) and the bacteria were resuspended in YEB medium. Tobacco leaves of a tobacco sterile culture (Nicotiana tabacum cv. Samsun NN) were cut into small approx. 1 cm²-sized pieces and bathed in the bacterial suspension. The leaf pieces were then placed on MS medium (0.7% agar) and incubated for 2 days in the dark. The leaf pieces were then placed on MS medium (0.7% agar) with 1.6% glucose, 1 mg/l benzylaminopurine, 0.2 mg/l naphthyl acetic acid, 500 mg/l Claforan and 50 mg/l kanamycin for shoot induction. The medium was changed every 7 to 10 days. If shoots developed, the leaf pieces were transferred to glass vessels which contained the same medium. Forming shoots were cut off and placed on MS medium+2% saccharose+250 mg/l Claforan and whole plants regenerated from them.

6. Determination of the Citrate Synthase Activity in Tissues of Transgenic Potato and Tobacco Plants and Non-transformed Potato and Tobacco Plants.

To determine the citrate synthase activity, raw extracts from tubers, leaves and flowers were produced and mitochondria isolated from potato tubers. To produce raw extracts, the material in question was frozen in liquid nitrogen, homogenized in extraction buffer (Neuhaus and Stitt (1990) Planta 182:445-454), centrifuged, and the supernatant liquid was then used for the activity test. To isolate mitochondria from potato tubers, 100-200 g of freshly harvested tubers were peeled and homogenized in 100 ml “grinding buffer” (0.4 M mannitol, 1 mM EDTA, 25 mM MOPS, 0.1% BSA, 10 mM b-mercaptoethanol, 0.05 mM PMSF, pH 7.8). The homogenate was filtered through 4 layers of cotton gauze and centrifuged for −4 min at 3500 g. The supernatant was filtered through 2 layers of “Miracloth” (Calbiochem) and centrifuged again for 30 min at 18000 g. The pellet was resuspended using a soft brush in 2 ml resuspension buffer (0.4 M mannitol, 20 mM Trizin, 2 mM EDTA, pH 7.2). After homogenizing twice in a “potter” homogenizer, the extract was coated onto a discontinuous Percoll gradient and centrifuged for 1 h at 72000 g. Mitochondria were removed from the 28%/45% interphase, washed and centrifuged twice for 15 min at 14500 g in “washing buffer” (0.4 M mannitol, 5 mM MOPS, 0.1% BSA, 0.2 mM PMSF, pH 7.5). The mitochondria were then resuspended in 100 μl resuspension buffer. To determine the citrate synthase activity 5 μl of the mitochondria suspension were taken up in 100 μl extraction buffer (Neuhaus and Stitt (1990) Planta 182:445-454). The citrate synthase activity was determined by means of spectrophotometry at 412 nm and 30° C. according to the Srere method (1967, Methods in Enzymology 13:3-22).

7. RNA Extraction and Northern Blot Experiments

RNA was isolated from frozen plant material as described in Logemann et al. (1987, Anal. Biochem. 163:21-26). The RNA was denatured in 40% formamide. The RNA was then separated by gel electrophoresis on formaldehyde/agarose gels, and after the gel run, blotted on nylon membrane (Hybond N; Amersham, UK). Hybridization with a radioactively-labelled DNA sample was carried out according to standard methods.

8. Plant Maintenance

Potato plants (Solanum tuberosum) were kept in a green house at 60% humidity and 22° C. for 16 h in the light and at 15° C. for 8 h in the dark. Tobacco plants (Nicotiana tabacum) were kept in the green house at 60% humidity and 25° C. for 14 h in the light and for 10 h at 20° C. in the dark.

### EXAMPLES

### Example 1

Cloning of a cDNA of the Citrate Synthase From Potato

To identify a cDNA from potato which codes for citrate synthase, a DNA fragment of the already-known cDNA of citrate synthase from Arabidopsis thaliana (Unger et al. (1989) Plant Mol. Biol. 13:411-418) was firstly amplified. For this, whole DNA was extracted from green plant tissue of Arabidopsis thaliana plants and poly(A⁺)-mRNA was prepared from this. This was then used for the preparation of cDNA. Using the oligodesoxynucleotides

(SeqID No. 4)

5′-AAGTGGATCCATGGTGTTTTTCCGCAGCGTAT-3′
and

(SeqID No. 5)

5′-CATAGGATCCTTAAGCAGATGAAGCTTTCTTA-3′,

which are complementary to the 5′- or 3′-end of the coding region of the cDNA of the citrate synthase from Arabidopsis thaliana (Unger et al. (1989) Plant Mol. Biol. 13: 411-418), a 1438 bp-long DNA fragment which codes for the citrate synthase from Arabidopsis thaliana was isolated from this cDNA preparation by a “polymerase chain reaction” (PCR). The oligonucleotides used additionally introduce BamHI cleavage sites at both ends of the amplified DNA fragment. The DNA fragment resulting from the PCR reaction was digested with BamHI and ligated into the plasmid PUC9.2 cleaved with BamHI. The cDNA insertion of this plasmid was later used as a heterologous sample for identifying a cDNA coding for citrate synthase from potato.

To produce a cDNA library, poly(A⁺)-mRNA was isolated from leaves of potato plants. Starting from the poly(A⁺)-mRNA, cDNA was produced which was provided with EcoRI/NotI-linkers and with which a cDNA library was placed in the vector Lambda ZAP II (Stratagene, USA) (KoBmann et al. (1992) Planta 188:7-12). 250000 plaques of this cDNA library were investigated using the heterologous sample from Arabidopsis thaliana for DNA sequences which are homologous to this. For this, the plaques were transferred onto nitrocellulose filters and denatured by NaOH treatment. The filters were then neutralized and the DNA fixed on the filters using heat treatment. The filters were pre-hybridized in 25% formamide, 0.5% BSA, 1% SDS, 5×SSC, 5×Denhardt solution, 40 mM sodium phosphate buffer pH 7.2 and 100 mg/ml salmon sperm DNA for 2 hours at 42° C. The filters were then hybridized overnight at 42° C. in 25% formamide, 0.5% BSA, 1% SDS, 5×SSC, 5×Denhardt solution, 40 mM sodium phosphate buffer pH 7.2 and 100 μg/ml salmon sperm DNA after adding the P³²-labelled cDNA coding for citrate synthase from Arabidopsis thaliana. The filters were washed for 30 min in 5×SSC, 0.5% SDS at 42° C. and for 20 min in 3×SSC, 0.5% SDS at 42° C. Phage clones of the cDNA library which hybridized with the cDNA used from Arabidopsis thaliana were further purified using standard processes. Using the in vivo excision method, E. coli clones which contain a double-stranded pBluescript plasmid with the corresponding cDNA insertion in the EcoRI cleavage site of the polylinker were obtained from positive phage clones. After checking the size and the restriction pattern of the insertions, a suitable clone was subjected to a sequence analysis.

### Example 2

Sequence Analysis of the cDNA Insertion of the Plasmid pPCS (DSM 8879)

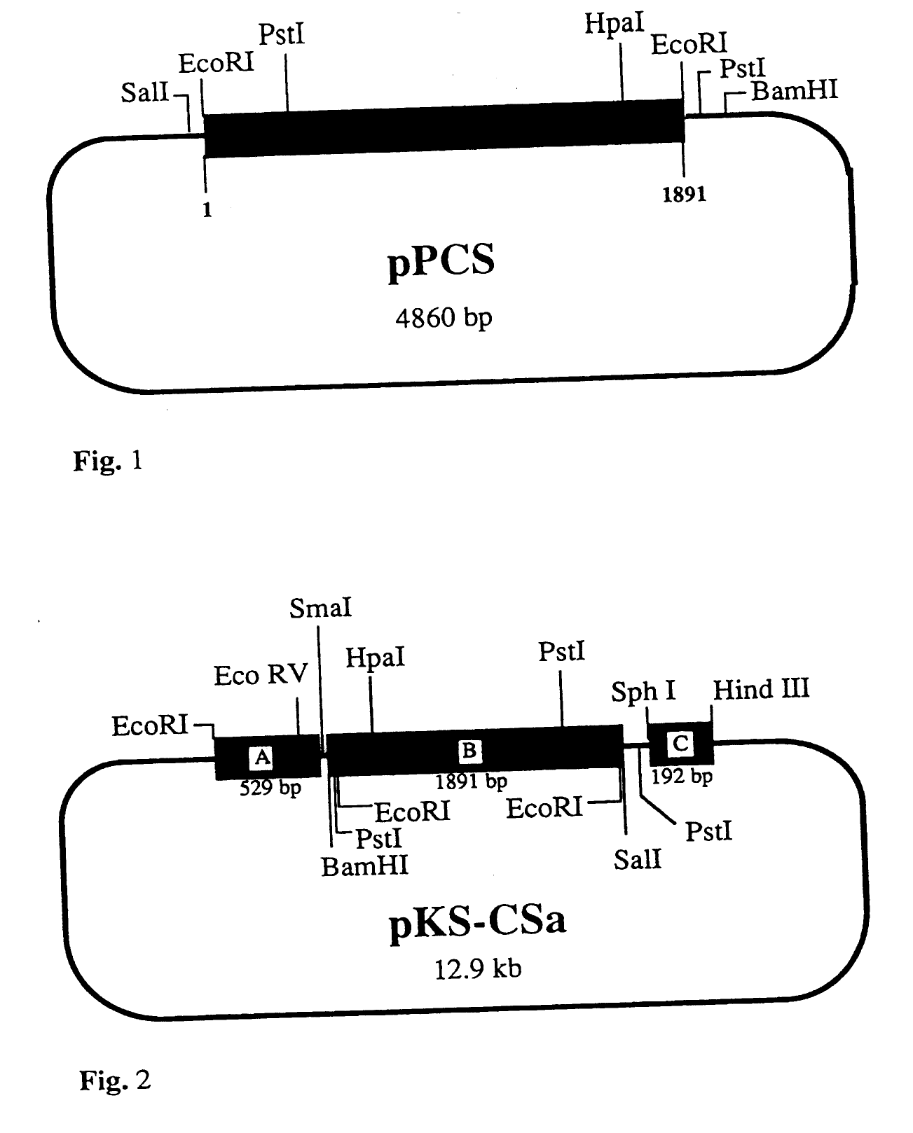

The plasmid pPCS (FIG. 1) was isolated from an E. coli clone obtained according to Example 1 and its cDNA insertion was determined by standard procedures using the didesoxy method (Sanger et al. (1977) Proc. Natl. Acad. Sci. USA 74:5463-5467). The insertion is 1891 bp long. The nucleotide sequence (SeqID No. 1) is given below.

### Example 3

Construction of the Plasmid pKS-CSa (DSM 8880) and Transfer of the Plasmid Into Potato Plants.

An approx. 1.9 kb long DNA fragment which has the sequence (Seq ID No. 1) given below and which contains the cloning region for citrate synthase from potatoes was isolated from the plasmid pPCS through BamHI/SalI digest. This DNA fragment was cloned into the vector pBinAR (Höfgen and Willmitzer (1990) Plant Sci. 66:221-230) cleaved using BamHI/SalI. The vector pBinAR is a derivative of the binary vector Bin19 (Bevan (1984) Nucleic Acids Res. 12:8711-8721). The resulting plasmid was called pKS-CSa and is shown in FIG. 2.

By inserting the cDNA fragment an expression cassette results which is constructed as follows from fragments A, B and C (FIG. 2):

Fragment A (529 bp) contains the 35S promoter of the cauliflower mosaic virus (CaMV). The fragment comprises the nucleotides 6909 to 7437 of the CaMV (Franck et al. (1980) Cell 21:285-294).

Fragment B comprises the protein-coding region of the citrate synthase from potatoes. This was isolated as described above as BamHI/SalI fragment from pPCS and fused to the promoter in pBinAR in anti-sense orientation.

Fragment C (192 bp) contains the polyadenylation signal of gene 3 of the T-DNA of the Ti-plasmid pTiACH5 (Gielen et al. (1984) EMBO J. 3:835-846).

The size of plasmid pKS-CSa is approx. 12.9 kb.

The vector pKS-CSa was transferred into potato plants using Agrobacterium tumefaciens-conveyed transformation. Intact plants were regenerated from the transformed cells. The result of the transformation was that transgenic potato plants showed to varying degree a reduction in the mRNA coding for the citrate synthase (see FIG. 6). 2 μg poly(A⁺)-mRNA were hybridized in a Northern Blot experiment with the probe for citrate synthase from potatoes. The transcript coding for citrate synthase which occurs in wild-type plants (lanes 1 to 3) is shorter than the transcript of the anti-sense expression cassette (see for example lane 6), from which it can be seen that the degree to which a reduction in the endogenous transcripts has occurred in the different transgenic plant varies.

Transgenic potato plants which show a reduction in the mRNA coding for the citrate synthase were investigated in different tissues for citrate synthase activity. The results of these investigations of leaves, tubers and mitochondria isolated from tubers are shown in the following table.

TABLE 1
Citrate synthase activity (in nmol/min/mg protein) in different
organs of the plants and in mitochondria

| | Wild type | T55 | T50 | T6 | T29 |
|---|---|---|---|---|---|
| Leaves | 55.6 ± 25.0 | 32.7 ± 25.0 | 15.1 ± 8.7 | 15.0 ± 7.7 | 3.2 ± 1.2 |
| | 100% | 58.8% | 27.1% | 27.0% | 5.8% |
| Tubers | 8.5 ± 3.4 | 4.9 ± 0.8 | 1.1 ± 0.3 | 1.6 ± 0.5 | 2.0 ± 0.8 |
| Mitochondria | 1788 ± 492 | 450 ± 120 | 265 ± 45 | 260 ± 50 | 193 ± 118 |
| | 100% | 25.2% | 14.8% | 14.5% | 9.3% |

Wild type = Solanum tuberosum cv. Désirée,
T55, T50, T6, T29 = independent, transgenic potato lines

Reducing the citrate synthase activity has a considerable effect on flower formation in the transgenic plants, the markedness of which depends on the extent of inhibition of the citrate synthase activity.

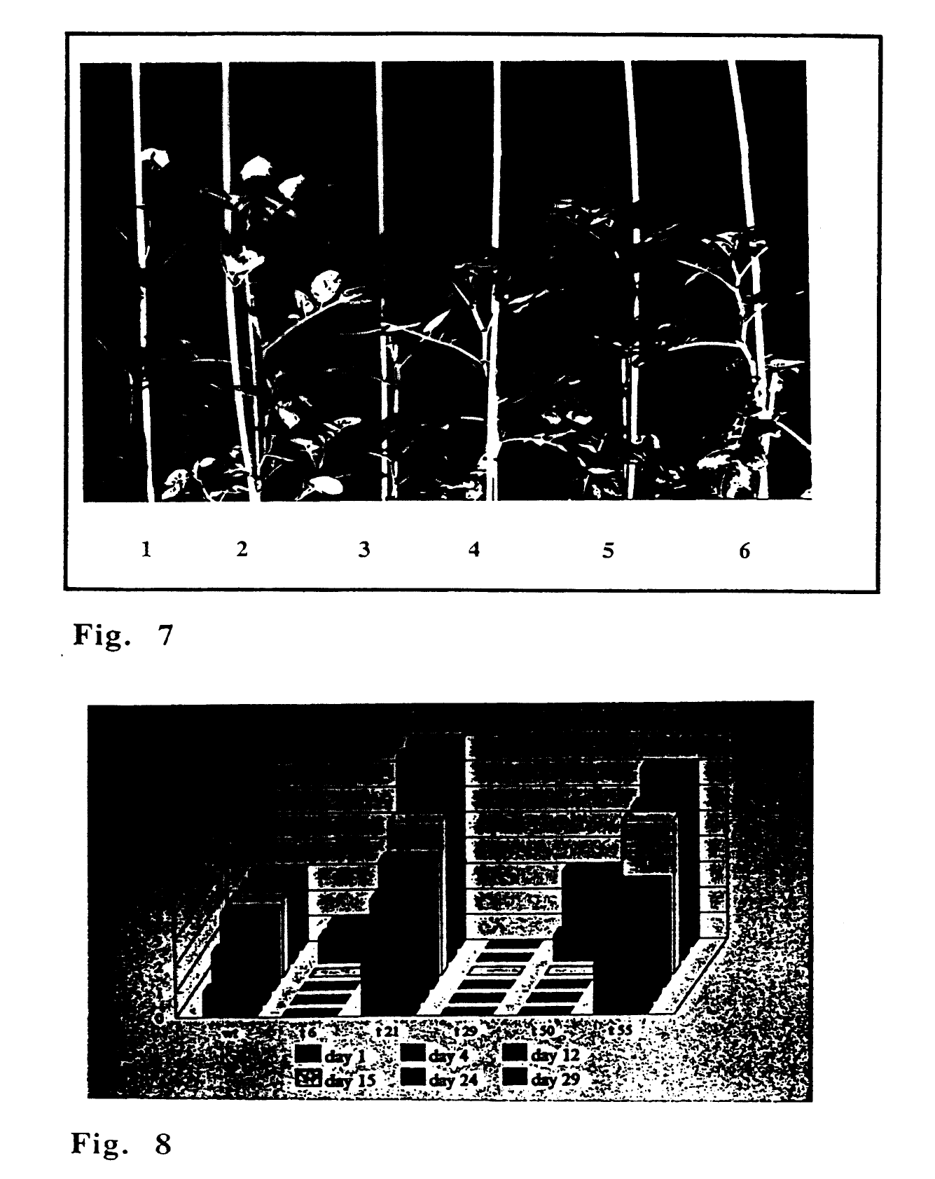

Transformed potato plants in which the citrate synthase activity is greatly reduced (see Table 1) are inhibited in their flower formation to a great extent or completely (see FIG. 7).

Plants in which the citrate synthase activity is only moderately reduced show delayed flower formation and produce fewer flowers or develop only flower buds which do not further develop to functional flowers, but die. This is shown in FIG. 8. Shown here are the number of plants with fully developed open flowers during one flowering period. 5 transgenic lines (T6, T21, T29, T50 and T55) are compared with wild-type plants. The transgenic line T21 is a transgenic line which displays no inhibition of the citrate synthase (100% citrate synthase activity). During the term of the investigation, no plant of the line T29 developed flowers and the plants of the lines T6 and T50 begin to flower only approx. 3 weeks later than wild-type plants.

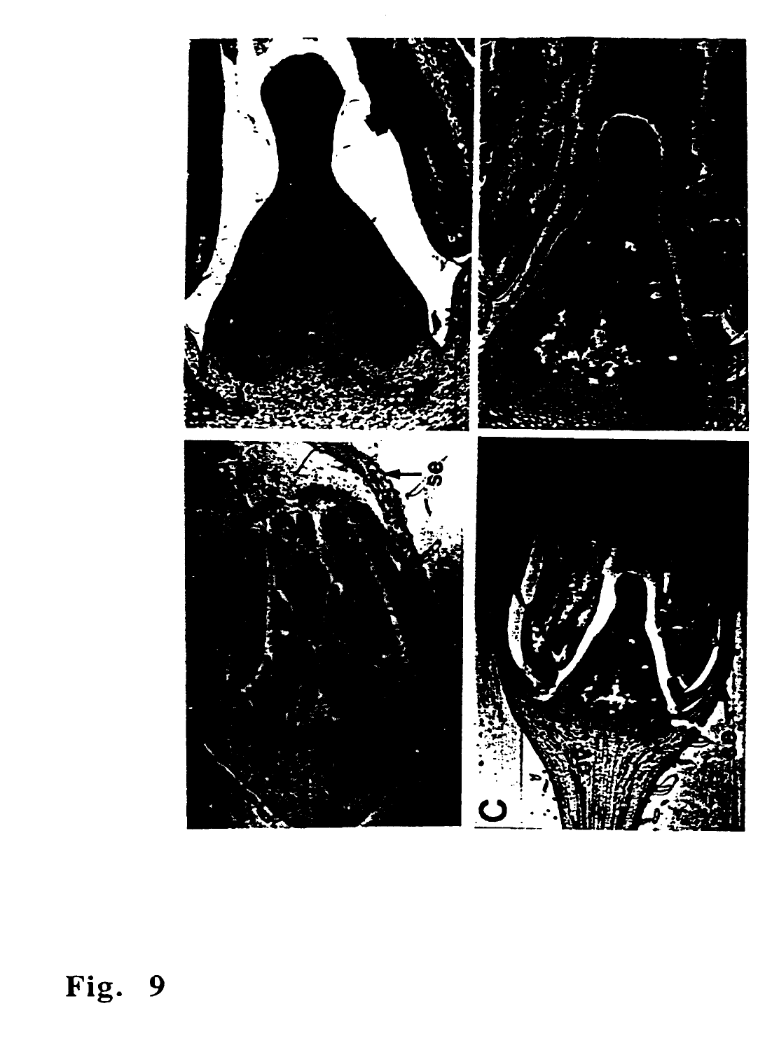

Other plants do develop flowers but these are not functional since the female reproductive organs (ovaries) are severely damaged. In these plants the ovaries disintegrate in the course of development. This is shown in FIG. 9. This figure shows longitudinal sections through flower buds of wild-type plants and transgenic plants of the line T29 in comparison. The tissues of the ovaries of transgenic plants are severely damaged compared with wild-type plants.

Using the present invention it is therefore also possible to produce plants according to the process according to the invention in which the citrate synthase activity is inhibited to varying degrees, so that from the transgenic plants can be chosen those which have the desired phenotype, for example a complete inhibition of flower formation, or flower formation whose onset, compared with non-transformed plants, is delayed, or which do develop buds from which, however, no functional flowers develop.

Reducing the citrate synthase activity also has a drastic effect on various properties of the tubers of the transformed potato plants. For example, tubers of transformed potato plants show lower storage losses after relatively long storage periods than tubers from non-transformed plants. This is expressed in a smaller loss of fresh or dry weight during the course of storage. The following table shows values for fresh and dry weights of tubers of transformed potato plants (line T6) and wild-type plants of the Desiree variety. The tubers were stored for 9 months at room temperature. The tuber weights are given in percentages, relative to the tuber fresh weights at the start of storage. The values are average values from 3 to 12 measurements with the standard deviation given. The values of the dry or fresh weights of the tubers of wild-type plants after 9 months' storage were taken as 100%.

TABLE 2

| | Wild type | TG |
|---|---|---|
| Tuber fresh weight | 68.7 ± 2.6 | 77.2 ± 1.3 |
| [%] | 100% | 112.4% |
| Tuber dry weight | 18.7 ± 2.6 | 21.7 ± 0.5 |
| [%] | 100% | 116% |

Wild type = Solanum tuberosum cv. Désirée,
T6 = transgeriic potato lines

The tubers of transformed potato plants also show a changed sprouting behaviour. The sprouts of these tubers, compared with tubers of wild-type plants, are substantially smaller and have a substantially lower fresh and dry weight. The following table shows values for fresh and dry weights of sprouts of tubers of transformed potato plants (line T6) and wild-type plants of the Desiree variety. The sprouts originate from tubers which were stored in the dark for 9 months at room temperature. The sprout weights are given in each case in grams. The values are average values from 3 to 12 measurements with the standard deviation given.

TABLE 3

| | Wild type | T6 |
|---|---|---|
| Sprouts | 2.1 ± 0.6 | 1.3 ± 0.4 |
| Fresh weight [g] | | |
| Sprouts | 0.31 ± 0.12 | 0.23 ± 0.06 |
| Dry weight [g] | | |

Wild type = Solanum tuberosum cv. Désirée,
TG = transgenic potato line

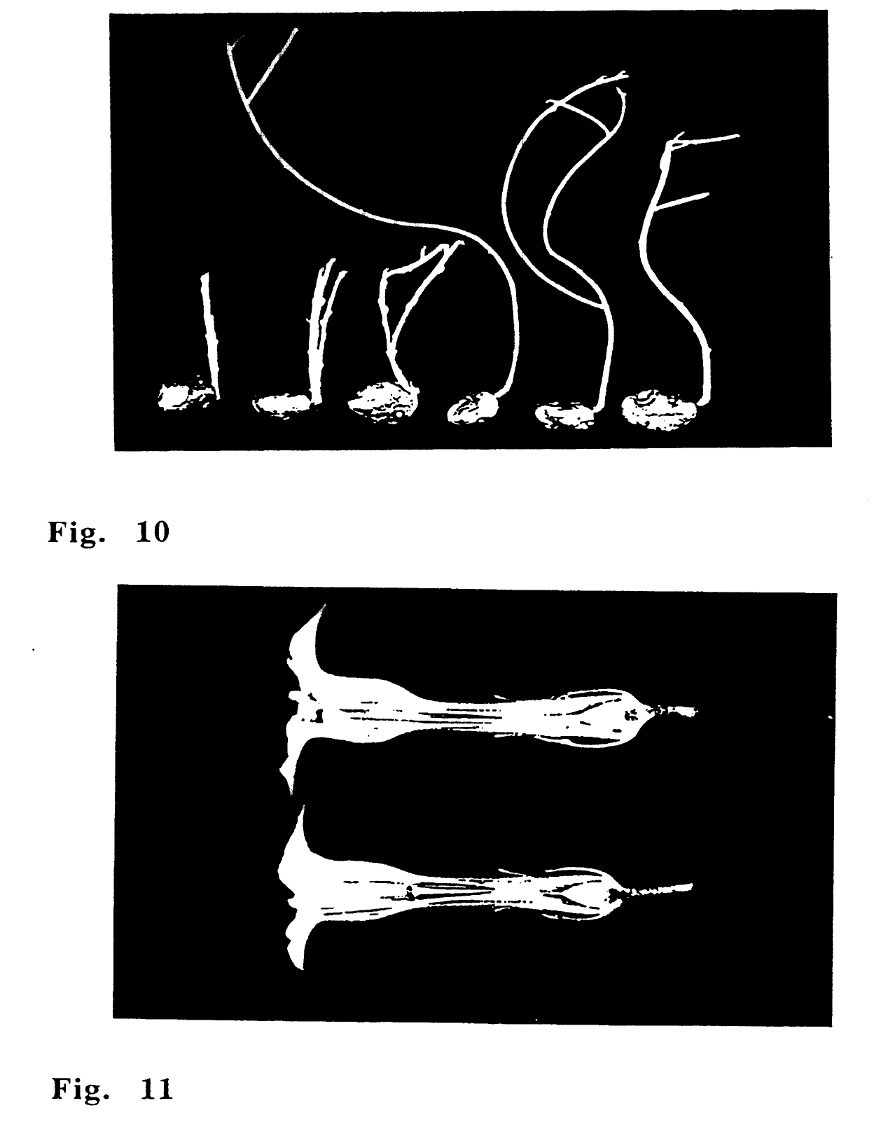

The modified sprouting behaviour is also illustrated by FIG. 10. Shown in each case are 3 tubers of the transformed potato line T6 and three tubers of a wild-type plant of the Désirée variety. The tubers were stored in the dark for 9 months at room temperature. The tubers of the transformed plants (left) form substantially smaller and shorter sprouts compared with the wild-type tubers (right).

### Example 4

Cloning of a cDNA Coding for Citrate Synthase From Tobacco (Nicotiana tabacum)

For the identification of a cDNA from Nicotiana tabacum which codes for citrate synthase, a cDNA bank of leaf tissue from tobacco was prepared as described in example 1 for potato. 250000 plaques of this cDNA bank were screened using a radioactive DNA probe for sequences which code for citrate synthase. The cDNA from Solanum tuberosum which codes for citrate synthase (1.4 kb NruI/HindII fragment from pPCS; see examples 1 and 2, and SeqID No. 1) was used as a probe. The identification and isolation of phage clones which hybridized with the radioactive DNA probe used took place as described in Example 1 with the difference that the plaques were transferred onto nylon membranes and the following buffer was used for the pre-hybridization and the hybridization: 0.25 M sodium phosphate buffer pH 7.2, 10 mM EDTA, 7% SDS, 10 mg BSA. Using the in vivo excision method, E. coli clones were obtained from positive phage clones which contain a double-stranded pBluescript plasmid with the cDNA insertion in question. After checking the size and the restriction pattern of the insertions, a suitable clone was subjected to a sequence analysis.

### Example 5

Sequence Analysis of the cDNA Insertion of the Plasmid pTCS (DSM 9357)

The plasmid pTCS (FIG. 4) was isolated from an E. coli clone obtained according to Example 4 and its cDNA insertion was determined by standard procedures using the didesoxy method (Sanger et al. (1977) Proc. Natl. Acad. Sci. USA 74: 5463-5467). The insertion is 1747 bp long. The nucleotide sequence is given below as SeqID No. 3.

### Example 6

Cloning of a cDNA Coding for Citrate Synthase from Sugar Beet (Beta vulgaris L.)

To identify a cDNA from sugar beet which codes for citrate synthase, a cDNA bank of leaf tissue from sugar beet (Beta vulgaris L. cultivated line 5S 0026) was prepared, isolating poly(A⁺)-RNA from leaf tissue and using this for the cDNA synthesis with the help of commercial kits (Pharmacia LKB, Stratagene, USA) according to the Gubler and Hoffmann method (1983, Gene 25:263-269). 250000 plaques of such a cDNA bank were screened as described in Example 4 using radioactive DNA probes for sequences which code for citrate synthase. Used as the probe was a mixture consisting of the radioactively labelled cDNA from Solanum tuberosum which codes for citrate synthase (see Examples 1, 2, and 4, and SeqID No. 1), and the radioactively-labelled cDNA from Nicotiana tabacum which codes for citrate synthase (see Examples 4 and 5, and SeqID No. 3). Phage clones which hybridized with the radioactive DNA sample used were identified and isolated as described in Example 1. Using the in vivo excision method, E. coli clones were obtained from positive phage clones which contain a double-stranded pBluescript plasmid with the cDNA insertion in question. After checking the size and the restriction pattern of the insertions, a suitable clone was subjected to a sequence analysis.

### Example 7

Sequence Analysis of the cDNA Insertion of the Plasmid pSBCS (DSM 9358)

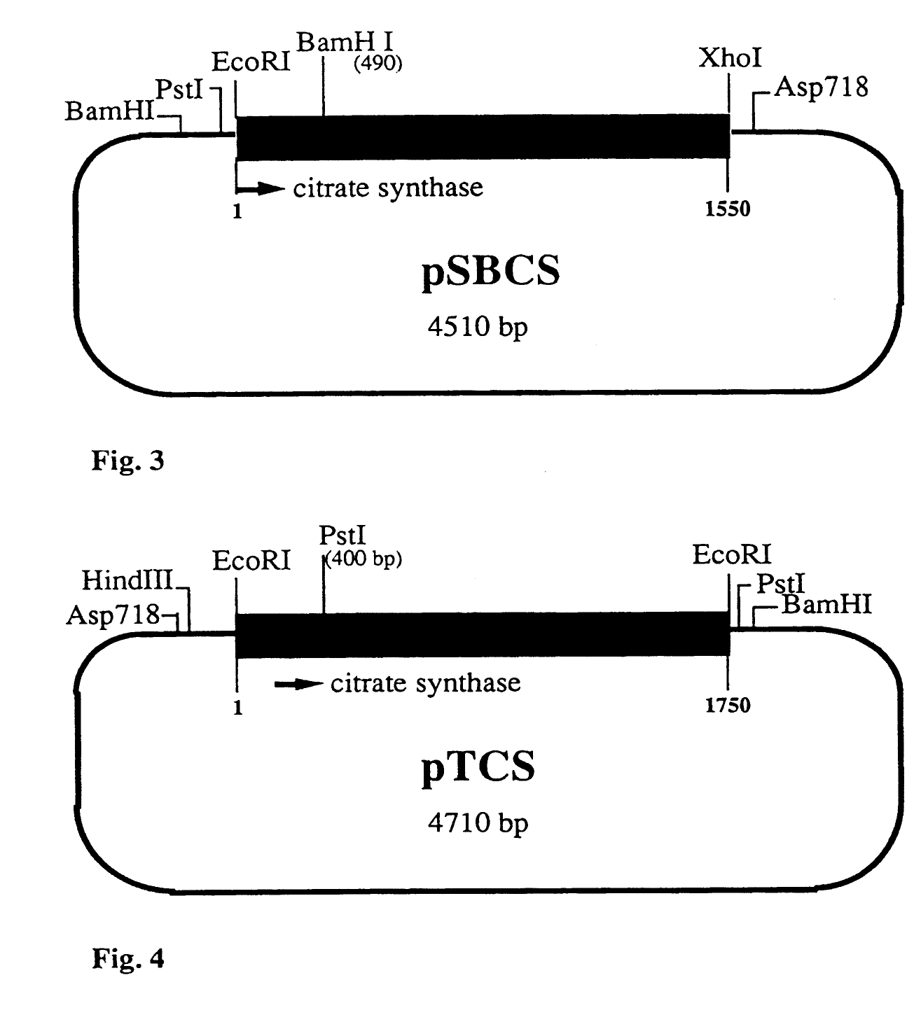

The plasmid pSBCS (FIG. 3) was isolated from an E. coli clone obtained according to Example 6 and its cDNA insertion was determined by standard procedures using the didesoxy method (Sanger et al. (1977) Proc. Natl. Acad. Sci. USA 74: 5463-5467). The insertion is 1551 bp long. The nucleotide sequence is given as SeqID NO. 2 below.

### Example 8

Construction of the Plasmid TCSAS (DSM 9359) and Transfer of the Plasmid Into Tobacco Plants.

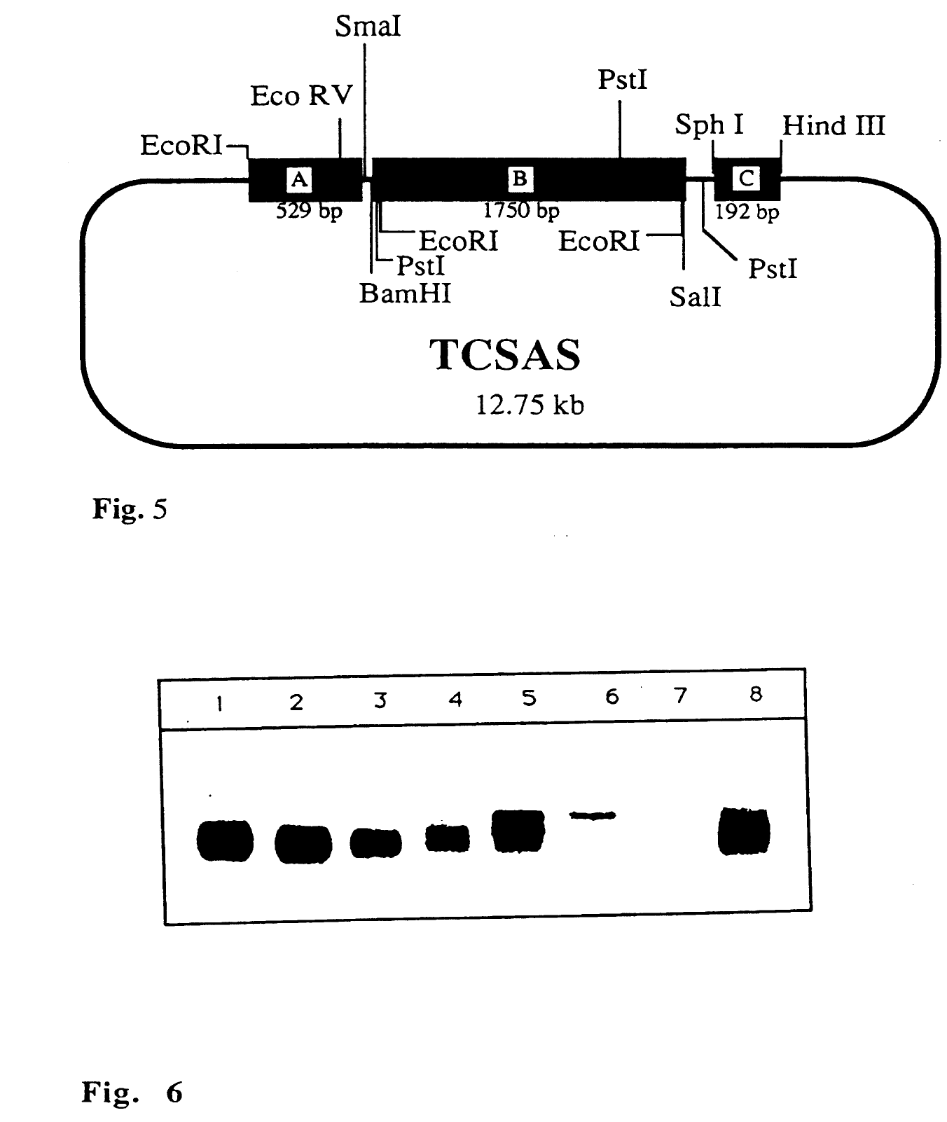

An approx. 1,800 kb-long DNA fragment, which has the sequence given below (SeqID No. 3) and which contains the coding region for citrate synthase from Nicotiana tabacum, was isolated from the plasmid pTCS by BamHI/SalI digest. This DNA fragment was cloned into the vector pBinAR cleaved with BamHI/SalI (Höfgen and Willmitzer (1990) Plant Sci. 66:221-230). The vector pBinAR is a derivative of the binary vector Bin19 (Bevan (1984) Nucleic Acids Res. 12:8711-8721). The resulting plasmid was called TCSAS and is shown in FIG. 5.

By inserting the cDNA fragment an expression cassette results which is constructed of the fragments A, B and C in the following way (FIG. 5):

Fragment A (529 bp) contains the 35S promoter of the cauliflower mosaic virus (CaMV). The fragment comprises the nucleotides 6909 to 7437 of the CaMV (Franck et al. (1980) Cell 21:285-294).

Fragment B contains, in addition to flanking regions, the protein-coding region of the citrate synthase from Nicotiana tabacum. This was isolated as described above as BamHI/SalI fragment from pTCS and fused in anti-sense orientation to the promoter in pBinAR.

Fragment C (192 bp) contains the polyadenylation signal of gene 3 of the T-DNA of the Ti-plasmid pTiACH5 (Gielen et al. (1984) EMBO J. 3:835-846).

The size of plasmid TCSAS is approx. 12.75 kb.

The plasmid was transferred into tobacco plants using agrobacteria-conveyed transformation as described above. Whole plants were regenerated from the transformed cells. The success of the genetic modification of the plants is tested by analyzing the whole RNA for the disappearance of the endogenous mRNA which codes for citrate synthase. Transgenic tobacco plants were investigated for citrate synthase activity in different tissues. The results of these investigations showed that, with the help of the process, tobacco plants can be produced in which the citrate synthase activity is reduced to varying degrees. As in the case of potato plants, different lines can therefore also be obtained with tobacco which differ as regards the extent of reduction in the citrate synthase activity.

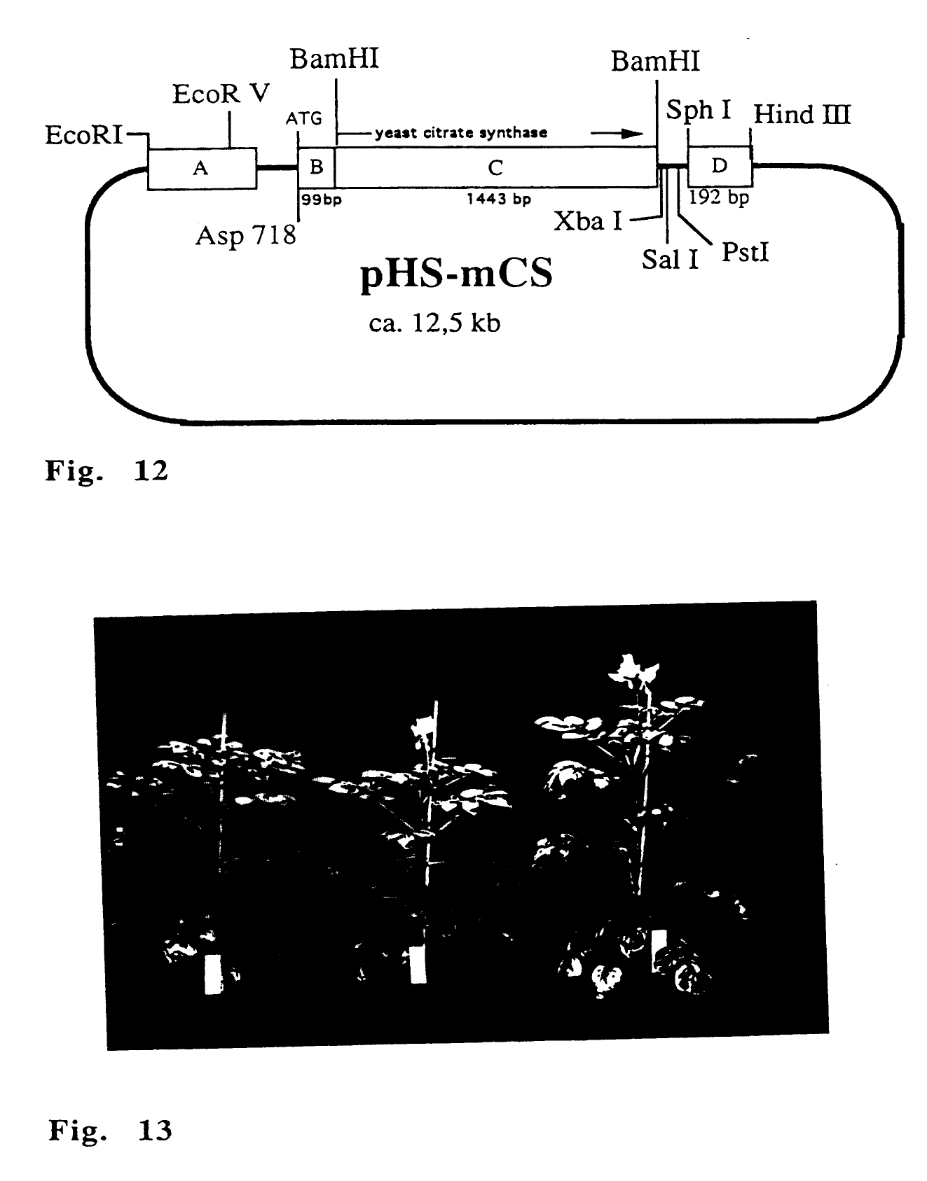

Also, in the case of tobacco plants, transformed plants showed a modified flowering behaviour. It is of particular interest that lines can be produced which produce flowers in which the pistil is severely shortened, compared with flowers of non-transformed plants. This is illustrated by FIG. 11 in which flowers of transformed and non-transformed tobacco plants are shown. This means that the inhibition of flower formation by reducing the citrate synthase activity both in tobacco and in the potato primarily affects the female flowering organs. These lines also display the phenotype, that they form substantially fewer seeds compared with wild-type plants, the quantity of seeds being determined with reference to the total weight of seeds formed.

### Example 9

Construction of the Plasmid pHS-mCS and Transfer of the Plasmid Into Potato Plants.

To construct the plasmid pHS-mCS, a DNA sequence which codes for the mitochondrial targeting sequence of the matrix processing peptidase (MPP) was firstly integrated into a pUC18 vector. This sequence was isolated by means of the polymerase chain reaction (PCR) from a pBluescript plasmid which contained the cDNA sequence of the MPP (Braun et al., 1992, EMBO J. 11:3219-3227) using the following oligonucleotides:

| | | |
|---|---|---|
| Oligo a: | 5′-GATC GGT ACC ATG TAC AGA TGC GCA TCG TCT-3′ | (SeqID No. 6) |
| and | | |
| Oligo a: | 5′-GTAC GGA TCC CTT GGT TGC AAC AGC AGC TGA-3′ | (SeqID No. 7) |

The resulting DNA fragment comprised the nucleotides 299 to 397 of the sequence shown in Braun et al (1992, EMBO J. 11:3219-3227), which codes for the matrix processing peptidase. An Asp 718 cleavage site was inserted at the 5′-end of the sequence by oligonucleotide a. Oligonucleotide b inserted a BamHI cleavage site at the 3′-end of the sequence. The DNA fragment obtained from the PCR was cleaved with Asp718 and BamHI and cloned into the vector pUC18 cleaved with Asp718 and BamHI. The resulting vector was called pMTP.

A DNA sequence from Saccharmoyces cerevisiae which codes for a citrate synthase was cloned into the plasmid pMTP behind the mitochondrial targeting sequence in the same reading frame,. For this, genomic DNA was prepared from yeast by current methods and a 1443 bp-long fragment which comprises the coding region for citrate synthase from yeast was isolated by means of PCR using the oligonucleotides

| | | |
|---|---|---|
| Oligo c: | 5′- CTAG GGA TCC ATG TCA GCG ATA TTA TCA ACA ACT AGC AAA AGT-3′ | (SeqID No. 8) |
| and | | |
| Oligo d: | 5′- GATT GGA TCC TTA GTT CTT ACT TTC GAT TTT CTT TAC CAA CTC-3′ | (SeqID No. 9) |

In particular, the sequence comprises the nucleotides 376-1818 of the sequence illustrated in Suissa et al. (1984, EMBO J. 3:1773-1781). The oligonucleotides used introduce a BamHI cleavage site on both sides of the amplified DNA sequence. The resulting DNA fragment was cleaved with the restriction endonuclease BamHI, then ligated into the vector pMTP cleaved with BamHI and transformed in E. coli cells. By determining the restriction pattern a clone was selected in which the insertion of the PCR fragment took place in such a way that the coding region was joined to the mitochondrial targeting sequence in sense orientation, i.e. such that the 5′-end of the coding region was joined to the 3′-end of the targeting sequence. The resulting plasmid was called pMTP-YCS.

Using the restriction endonucleases Asp718 and Xba I, an approx. 1550 bp-long fragment which comprises the mitochondrial targeting sequence and the coding region for citrate synthase from yeast was isolated from the vector pMTP-YCS. This fragment was ligated into the binary vector pBinAR (Höfgen and Willmitzer, 1990, Plant Sci. 66: 221-230) cleaved with Asp718 and Xba I. The resulting plasmid pHS-mCS is shown in FIG. 12. The binary vector pBinAR is a derivative of the binary vector Bin19. The vector contains a 35S promoter and a termination signal for the transcription, between which is located a polylinker which can be used for inserting various DNA sequences.

By inserting the DNA fragment which codes for citrate synthase from yeast with a mitochondrial target sequence at the N-terminus, an expression cassette results which is constructed of fragments A, B and C in the following manner (FIG. 12):

Fragment A (529 bp) contains the 35 S promoter of the cauliflower mosaic virus (CaMV). The fragment comprises the nucleotides 6909 to 7437 of the CaMV (Franck et al. (1980) Cell 21:285-294).

Fragment B contains a 99 bp-long DNA fragment which codes for the mitochondrial target sequence of the matrix processing peptidase (nucleotides 299-397 of the sequence shown in Braun et al., 1992, EMBO J. 11:3219-3227).

Fragment C contains the coding region for citrate synthase from Saccharomyces cerevisiae (nucleotides 376-1818 of the sequence shown in Suissa et al., 1984 EMBO J. 3:1773-1781) fused in sense orientation and in the same reading frame as the target sequence to the 3′-end of the target sequence.

Fragment D (192 bp) contains the polyadenylation signal of gene 3 of the T-DNA of the Ti plasmid pTiACH5 (Gielen et al. (1984) EMBO J. 3:835-846).

The size of the plasmid pHS-mCS is approx. 12.5 kb.

From this expression cassette, a transcript is transcribed by the 35S promoter which codes for a citrate synthase from yeast and comprises at its N-terminus an amino acid sequence which ensures transportation of the protein into the mitochondria.

The plasmid was transferred into potato plants using agrobacteria-conveyed transformation as described above. Whole plants were regenerated from the transformed cells. The result of the transformation was that transgenic potato plants showed an expression of the yeast citrate synthase in the cells. This was demonstrated with the help of Western Blot analyses using polyclonal antibodies which specifically recognise the citrate synthase from yeast. The transformed potato plants which showed a high expression of the citrate synthase from yeast display a modified flowering behaviour compared with non-transformed potato plants. On the one hand it was to be observed that transformed plants start to produce flowers substantially earlier (under green house conditions, on average 2-4 weeks) and produced more flowers compared with non-transformed plants. The premature flower formation of the transgenic potato plants is illustrated in FIG. 13. This shows two transgenic potato plants which had been transformed with plasmid pHS-mCS, compared with a wild type plant of the Desiree variety. The transgenic plants also produced substantially more flowers. In particular, after the first inflorescence had faded, the transgenic plants as a rule developed a second inflorescence and in some cases even a third inflorescence. In contrast, wild-type plants have only one florescence and die when this inflorescence has faded.

### Example 10

Construction of the Plasmid pEC-mCS and Transfer of the Plasmid Into Potato Plants.

To produce the plasmid pEC-mCS, a DNA sequence from E. coli which codes for a citrate synthase was cloned into the plasmid pMTP described in Example 9 behind the mitochondrial targeting sequence in the same reading frame. For this, genomic DNA was prepared from E. coli DH5α by current methods and an approx. 1280 bp-long fragment which comprises the coding region for citrate synthase from E. coli was isolated by means of PCR using the oligonucleotides

(SeqID No. 10)

| | |
|---|---|
| Oligo e: | 5′- GTAGGGATCC ATGGCTGATA CAAAAGCAA - 3′ |
| and | |

(SeqID No. 11)

| | |
|---|---|
| Oligo f: | 5′- GATTGGATCCTTAACGCTTGATATCGCTT - 3′ |

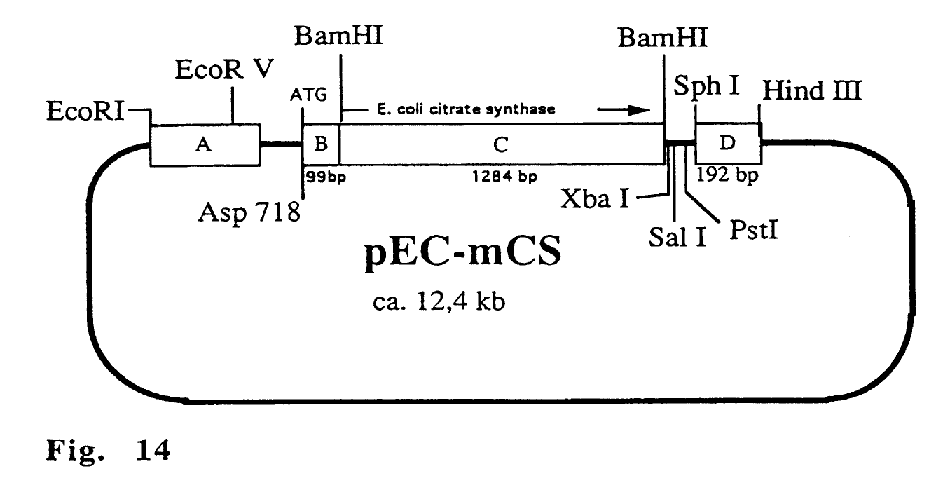

The sequence comprises in particular the nucleotides 306-1589 of the sequence illustrated in Sarbjit et al. (1983, Biochemistry. 22:5243-5249). The oligonucleotides used introduce a BamHI cleavage site at both sides of the amplified DNA sequence. The resulting DNA fragment was cleaved with the restriction endonuclease BamHI, then ligated into the vector pMTP cleaved with BamHI and introduced into E. coli cells by transformation. By determining the restriction pattern, a clone was selected in which the insertion of the PCR fragment took place in such a way that the coding region was joined to the mitochondrial targeting sequence in sense orientation, i.e. that the 5′-end of the coding region was joined to the 3′-end of the targeting sequence. The resulting plasmid was called pMTP-ECCS. Using restriction endonucleases Asp718 and Xba I a fragment was isolated from this vector which comprises the mitochondrial targeting sequence and the coding region for citrate synthase from E. coli. This fragment was ligated into the binary vector pBinAR cleaved with Asp718 and Xba I (Höfgen and Willmitzer, 1990, Plant Sci. 66:221-230). The resulting plasmid pEC-mCS is illustrated in FIG. 14.

By inserting the DNA fragment which codes for citrate synthase from E. coli with a mitochondrial targeting sequence at the N-terminus, an expression cassette results which is constructed from the fragments A, B, C and D in the following manner (FIG. 14):

Fragment A (529 bp) contains the 35 S promoter of the cauliflower mosaic virus (CaMV). The fragment comprises the nucleotides 6909 to 7437 of the CaMV (Franck et al. (1980) Cell 21:285-294).

Fragment B contains a 99 bp-long DNA fragment which codes for the mitochondrial targeting sequence of the matrix processing peptidase (nucleotides 299-397 of the sequence shown in Braun et al., 1992, EMBO J. 11:3219-3227).

Fragment C contains the coding region for citrate synthase from E. coli (nucleotides 306-1589 of the sequence shown in Sarbjit et al., 1983, Biochemistry. 22:5243-5249) fused in sense orientation and in the same reading frame as the targeting sequence to the 3′-end of the targeting sequence.

Fragment D (192 bp) contains the polyadenylation signal of gene 3 of the T-DNA of the Ti plasmid pTiACH5 (Gielen et al. (1984) EMBO J. 3:835-846).

The size of the plasmid pEC-mCS is approx. 12.4 kb.

From this expression cassette, a transcript is transcribed by the 35S promoter which codes for a citrate synthase from E. coli and comprises at its N-terminus an amino acid sequence which ensures transportation of the protein into the mitochondria.

The plasmid was transferred into potato plants using agrobacteria-conveyed transformation as described above. Whole plants were regenerated from the transformed cells and analyzed for citrate synthase activity.

1. A transgenic plant comprising transgenic plant cells with a reduced citrate synthase activity in comparison to wild type cells, said plant displaying inhibition of flower formation.

2. The transgenic plant of claim 1, wherein the citrate synthase activity is reduced by inhibiting the expression of DNA sequences which code for citrate synthase.

3. The transgenic plant of claim 2, wherein the inhibition of expression is achieved by the use of antisense RNA.

4. The transgenic plant of claim 2, wherein the inhibition of expression is achieved by the use of ribozymes cleaving specifically RNA coding for citrate synthase.

5. The transgenic plant of any one of claims 1 to 4 which is a useful plant.

6. A storage organ of a plant of any one of claims 1 to 5 comprising cells with a reduced citrate synthase activity.

7. The storage organ of claim 6 which is a tuber.

8. A transgenic plant comprising plant cells which have an increased citrate synthase activity compared to wild type cells because of the additional expression of a DNA sequence which codes for a protein having the enzymatic activity of a citrate synthase, said plant displaying a modified flowering behaviour. [symbol]
(ii) a DNA sequence coding for citrate synthase which is fused to the promoter in sense orientation.

17. A vector containing a DNA molecule of claim 15 or 16.

18. Plasmid pKS-CSa (DSM 8880).

19. Plasmid TCSAS (DSM 9359).

20. Bacteria, containing a DNA molecule of claim 15 or 16 or a vector of any one of claims 17 to 19.

21. A process for inhibiting flower formation in plants wherein the citrate synthase activity in the cells of the plants is reduced.

22. A process to improve the storage capability of storage organs in plants wherein the citrate synthase activity in the cells of the plants is reduced.

23. A process for reducing the sprouting of tubers of transgenic tuberous plants wherein the citrate synthase activity in the cells of the tubers is reduced.

24. The process of any one of claims 21 to 23, wherein the citrate synthase activity is reduced by inhibiting the expression of DNA sequences which code for citrate synthase.

25. The process of claim 24, wherein the expression of DNA sequences which code for citrate synthase is inhibited by the use of anti-sense RNA.

26. The process of claim 25 wherein
(a) a DNA which is complementary to a citrate synthase gene present in the cell is stably integrated into the gene of a plant cell;
(b) this DNA is expressed constitutively or upon induction due to the combination with suitable elements controlling the transcription;
(c) the expression of endogenous citrate synthase genes is inhibited because of an anti-sense effect; and
(d) plants are regenerated from the transgenic cells.

27. The process of claim 25 or 26, wherein the DNA sequence transcribed into anti-sense RNA comprises a nucleotide sequence which codes in sense orientation for a protein having the amino acid sequence given in SeqID No. 1 or SeqID No. 2 or SeqID No. 3 or a DNA sequence which shows a high degree of homology to such a DNA sequence or a part of such sequence wherein the used DNA sequence or part thereof has a length ad a degree of homology to an endogenous citrate synthase gene sufficient to elicit an antisense effect and thereby inhibit expression of said endogenous citrate synthase gene.

28. The process of any one of claims 25 to 27, wherein the DNA sequence transcribed into anti-sense RNA comprises the nucleotide sequence given in SeqID No. 1 or SeqID No. 2 or SeqID No. 3, an essentially identical nucleotide sequence or a part thereof or derivatives thereof which are derived by insertion, deletion or substitution of this sequence or a DNA sequence which shows a high degree of homology to such a DNA sequence or a part of such sequence wherein the used DNA sequence or part thereof has a length and a degree of homology to an endogenous citrate synthase gene sufficient to elicit an antisense effect and thereby inhibit expression of said endogenous citrate synthase gene.

29. The process of claim 24, wherein the expression of DNA sequences which code for citrate synthase is inhibited by use of ribozymes.

30. A process for inducing flower formation in plants, wherein the citrate synthase activity in the cells of the plant is increased.

31. The process of claim 30, wherein the increase in citrate synthase activity is achieved by expression of a recombinant DNA molecule which is stably integrated into the genome of the plant cells and which comprises the coding region for a citrate synthase and leads to the expression of a citrate synthase in the transformed cells.

32. The process of claim 31, wherein
(a) DNA which is of homologous or heterologous origin and which codes for a protein having a citrate synthase activity is stably integrated into the genome of a plant cell;
(b) this DNA is expressed constitutively or upon induction due to the combination with suitable elements controlling the transcription;
(c) because of this expression the citrate synthase activity in the transgenic cells increases and
(d) plants are regenerated form the transgenic cells.

33. The process of claim 31 or 32, wherein the DNA sequence comprises a nucleotide sequence which codes for a protein having the amino acid sequence given in SeqID No. 1 or SeqID No. 2 or SeqID No. 3 or an essentially identical amino acid sequence or for a part of these sequences wherein the protein encoded by the DNA sequence or the part thereof displays citrate synthase activity.

34. The process of claim 31 or 32, wherein the DNA sequence comprises the nucleotide sequence given in SeqID No. 1 or SeqID No. 2 or SeqID No. 3 or an essentially identical nucleotide sequence or a part thereof, wherein the protein encoded by the DNA sequence or part thereof displays citrate synthase activity.

35. The process of claim 31 or 32, wherein the DNA sequence codes for a deregulated or unregulated citrate synthase.

36. The process of claim 35 wherein the DNA sequence originates from Saccharomyces cerevisae.

37. The process of claim 35 wherein the DNA sequence originates from a prokaryotic organism.

38. The process of claim 37 wherein the procaryotic organism is E. coli.

39. Use of DNA sequences which code for citrate synthase (EC No. 4.1.3.7.) for modifying the flowering behaviour of plants.

40. The use of claim 39 wherein the flower formation is inhibited.

41. The use of claim 39 wherein the flower formation is induced.

42. A DNA sequence of a plant of the Solanaceae family or the Chenopodiaceae family which contains the coding region for a citrate synthase (EC No. 4.1.3.7.), characterized in that the information contained in the nucleotide sequence permits, upon integration into a plant genome, the formation of transcripts through which an endogenous citrate synthase activity can be suppressed, or permits the formation of transcripts by which the citrate synthase activity in the cells can be increased.

43. The DNA sequence of claim 1, which originates from a plant of the species Solanum tuberosum.

44. The DNA sequence of claim 1, which originates from a plant of the species Nicotiana tabacum.

45. The DNA sequence of claim 1, which originates from a plant of the species sugar beet (Beta vulgaris).

46. The DNA sequence of claim 1, which codes for a protein comprising the amino acid sequence given in SeqID No. 1 or an essentially identical amino acid sequence, said protein having citrate synthase activity.

47. The DNA sequence of claim 1, which codes for a protein comprising the amino acid sequence given in SeqID No. 2 or and essentially identical amino acid sequence, said protein having citrate synthase activity.

48. The DNA sequence of claim 1, which codes for a protein comprising the amino acid sequence given in SeqID No. 3 or an essentially identical amino acid sequence, said protein having citrate synthase activity.

49. The DNA sequence of claim 1, which comprises the nucleotide sequence given in SeqID No. 1 or an essentially identical nucleotide sequence which codes for a protein having citrate synthase activity.

50. The DNA sequence of claim 1, which comprises the nucleotide sequence given in SeqID No. 3 or an essentially identical nucleotide sequence which codes for a protein having citrate synthase activity.

51. The DNA sequence of claim 1, which comprises the nucleotide sequence given in SeqID No. 2 or an essentially identical nucleotide sequence which codes for a protein having citrate synthase activity.

52. A plasmid comprising a DNA sequence of any one of claims 42 to 51.

53. Plasmid pPCS (DSM 8879).

54. Plasmid pSBCS (DSM 9385).

55. Plasmid pTCS (DSM 9357).

56. Bacteria containing a DNA sequence of any one of claims 42 to 51 or a plasmid of any one of claims 52 to 55.

57. Use of a DNA sequence of any one of claims 42 to 51 in combination with control elements for an expression in pro- and eucaryotic cells.

58. Use of a DNA sequence of any one of claims 42 to 51 for the expression of a non-translatable mRNA which prevents the synthesis of an endogenous citrate synthase in the cells.

59. Use of a DNA sequence of any one of claims 49 to 51 for isolating homologous sequences from the genome of plants.
